(19) United States
(12) Patent Application Publication
(10) Pub. No.: US 2019/0034797 A1
(43) Pub. Date: Jan. 31, 2019

(54) DATA GENERATION APPARATUS, BIOLOGICAL DATA MEASUREMENT SYSTEM, CLASSIFIER GENERATION APPARATUS, DATA GENERATION METHOD, CLASSIFIER GENERATION METHOD, AND RECORDING MEDIUM

(71) Applicant: Panasonic Intellectual Property Management Co., Ltd., Osaka (JP)

(72) Inventors: ASUKA SAKAI, Tokyo (JP); YUKI MINODA, Tokyo (JP); KOJI MORIKAWA, Tokyo (JP)

(21) Appl. No.: 16/042,122

(22) Filed: Jul. 23, 2018

(30) Foreign Application Priority Data
Jul. 28, 2017 (JP) 2017-147165

(51) Int. Cl. G06N 3/08; G06N 3/06; A61B 5/16; A61B 5/00; G06F 3/01

(52) U.S. Cl. CPC G06N 3/08; G06N 3/06; G06F 3/015; A61B 5/4064; A61B 5/165

(57) ABSTRACT

A data generation apparatus includes a processor and a memory. The memory stores a generation rule including one or more time change values and/or one or more amplitude change values. The processor (a1) obtains a first biological data item including an event-related potential of a user and obtains a first label associated with the first biological data item, (a2) refers to the generation rule and generates one or more second biological data items by changing the first biological data item using the one or more time change values and/or the one or more amplitude change values, and (a3) outputs the one or more second biological data items associated with the first label, as training data related to the user.

### BACKGROUND

### 1. Technical Field

The present disclosure relates to a data generation apparatus, a biological data measurement system, a classifier generation apparatus, a data generation method, a classifier generation method, and a recording medium for generating data items used in machine learning.

### 2. Description of the Related Art

Machine learning notably deep learning attracts attention as a method for highly accurately realizing complex classification and recognition that have hitherto been difficult. A necessary condition for efficient machine learning is preparation of a sufficient amount of training data. For example, in the field of image recognition, a recognition accuracy exceeding those achieved by conventional techniques is realized by using several tens of thousands to several hundreds of millions of images as training data in deep learning.

For example, Japanese Unexamined Patent Application Publication No. 2006-130121 (hereinafter, PTL 1) discloses machine learning that uses biological information obtained from a body of a person. Specifically, PTL 1 discloses an apparatus that recognizes a feeling of a user by using biological information of the user. This recognition apparatus learns relationships between biological information and feeling information in advance and recognizes a feeling of a user by using the result of learning. In addition, Ciresan et al., “Deep Big Simple Neural Nets Excel on Handwritten Digit Recognition”, Neural Computation, December 2010, Vol. 22, No. 12 (hereinafter, referred to as NPL 1) discloses a method for creating pseudo training data items, that is, pseudo data items used in learning, by performing parametric deformation on each image data item. Examples of the parametric deformation include luminance adjustment, inversion, distortion, enlargement, and reduction of an image.

### SUMMARY

In the case of generating pseudo biological training data items and of using such pseudo biological training data items in machine learning, it is difficult to use the method for creating pseudo training data items described in NPL 1 for the recognition apparatus described in PTL 1. A reason for this is that data items targeted by NPL 1 and data items targeted by PTL 1 have different characteristics.

One non-limiting and exemplary embodiment provides a data generation apparatus, a biological data measurement system, a classifier generation apparatus, a data generation method, a classifier generation method, and a recording medium for generating pseudo biological data items from an existing biological data item.

In one general aspect, the techniques disclosed here feature a data generation apparatus including a memory that stores a generation rule including one or more time change values and/or one or more amplitude change values; and a processor that (a1) obtains a first biological data item including an event-related potential of a user and obtains a first label associated with the first biological data item, (a2) refers to the generation rule and generates one or more second biological data items by changing the first biological data item using the one or more time change values and/or the one or more amplitude change values, and (a3) outputs the one or more second biological data items associated with the first label, as training data related to the user.

The technique according to the present disclosure enables generation of pseudo biological data items from an existing biological data item.

It should be noted that general or specific embodiments may be implemented as a system, an apparatus, a method, an integrated circuit, a computer program, a computer-readable recording medium such as a recording disc, or any selective combination thereof. Examples of the computer-readable recording medium include a non-volatile recording medium, for example, a Compact Disc-Read Only Memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating a functional configuration of a data generation apparatus according to a first embodiment;

FIG. 2 is a table illustrating an example of information stored in an original data accumulating unit;

FIG. 3 is a flowchart illustrating an example of a flow of an operation performed by the data generation apparatus according to the first embodiment;

FIG. 4 is a flowchart illustrating an example of a flow of a detailed operation performed by a time shift operation unit according to the first embodiment;

FIG. 5 is a graph illustrating an example of waveform data items of brainwaves;

FIG. 6 is a graph illustrating an example of waveform data items of brainwaves generated by the time shift operation unit;

FIG. 7 is a flowchart illustrating an example of a flow of a detailed operation performed by an amplitude operation unit according to the first embodiment;

FIG. 8 is a graph illustrating an example of waveform data items of brainwaves generated by the amplitude operation unit;

FIG. 9 is a diagram illustrating a functional configuration of a classification system according to a second embodiment;

FIG. 10 is a flowchart illustrating an example of a flow of an operation performed when a classification apparatus according to the second embodiment learns from data items;

FIG. 11 is a flowchart illustrating an example of a flow of an operation performed when the classification apparatus according to the second embodiment recognizes a data item;

FIG. 12A is a diagram illustrating an application example of the classification system according to the second embodiment;

FIG. 12B is a diagram illustrating another application example of the classification system according to the second embodiment;

FIG. 13 is a diagram illustrating an application example of the classification apparatus according to the second embodiment;

FIG. 14 is a diagram illustrating a functional configuration of a data generation apparatus according to a third embodiment;

FIG. 15A is a graph illustrating an example of waveform data items of brainwaves;

FIG. 15B is a graph illustrating an example of a trial average waveform data item of the waveform data items illustrated in FIG. 15A;

FIG. 15C is a graph illustrating an example of waveform data items generated by changing a waveform data item illustrated in FIG. 15A in a change interval with respect to time;

FIG. 15D is a graph illustrating an example of waveform data items generated by changing a waveform data item illustrated in FIG. 15A in a change interval with respect to amplitude;

FIG. 16A is a diagram illustrating a data assignment method used when a classifier of a first comparative example is generated;

FIG. 16B is a diagram illustrating a data assignment method used when a classifier of a second comparative example is generated;

FIG. 16C is a diagram illustrating a data assignment method used when a classifier according to an example is generated;

FIG. 17 is a graph illustrating classification rates of the classifiers according to the first to third comparative examples;

FIG. 18 is a graph illustrating classification rates of the classifiers according to the example and the second comparative example;

FIG. 19 is a graph illustrating classification rates of the classifiers according to the example and the first to third comparative examples;

FIG. 20 is a graph illustrating a relationship between an amount by which data items are increased by the data generation apparatus and the classification rate of the classifier;

FIG. 21 is a graph illustrating a relationship between a time change value for a waveform data item and the classification rate of the classifier in the case where the data generation apparatus increases the amount of brainwave data items threefold;

FIG. 22 is a graph illustrating a relationship between an amplitude change value for a waveform data item and the classification rate of the classifier in the case where the data generation apparatus increases the amount of brainwave data items threefold;

FIG. 23 is a graph illustrating a relationship between a time change value for a waveform data item and the classification rate of the classifier in the case where the data generation apparatus increases the amount of brainwave data items threefold; and

FIG. 24 is a graph illustrating a relationship between an amplitude change value for a waveform data item and the classification rate of the classifier in the case where the data generation apparatus increases the amount of brainwave data items threefold.

### DETAILED DESCRIPTION

### Definition of Terms

First, definitions of terms used herein will be described. The term “biological data item” refers to a signal (also called a “biosignal”) emitted from a body due to a biological phenomenon, such as a signal of heartbeat, myoelectric potential, eye potential, or brainwave. The biological data item is represented in time series. A data item represented in time series includes data items that are measured at multiple time points and associate measured values and the respective time points with each other. A data item represented in time series may include sets (ti, M(ti)) of a time point ti and a measured value M(ti) measured at the time point ti, where 1≤i<n and i and n are natural numbers.

The term “event-related potential (ERP)” refers to fluctuations in a bioelectric potential caused by a response to the occurrence of an event that stimulates a person. A bioelectric potential is one of biosignals. Examples of measurable bioelectric potentials include an electroencephalogram (EEG), an electrocardiogram, an electrooculogram, or an electromyogram.

The term “latency” refers to a period from a time point at which a stimulus (for example, a visual stimulus or an auditory stimulus) that originates an event-related potential is presented to a time point at which a peak potential of a positive component or a negative component of the event-related potential appears. The term “peak” refers to a local maximum value or a local minimum value in the waveform of the potential.

The term “negative component” generally refers to a potential lower than 0 V. In the case where there is a target potential to be compared, the negative component includes a potential having a negative value relative to the value of the target potential, that is, a potential having a value less than the value of the target potential. The term “positive component” generally refers to a potential higher than 0 V. In the case where there is a target potential to be compared, the positive component includes a potential having a positive value relative to the value of the target potential, that is, a potential having a value greater than the value of the target potential.

Herein, a time point after a predetermined period has passed from a certain time point is sometimes expressed as, for example, “a latency of approximately 100 ms” in order to define the time component of the event-related potential. This expression indicates that the latency can include time points within a particular range centered at a particular time point of 100 ms. For example, according to Table 1 on page 30 of Kimitaka Kaga (editor) et al., “Jisho Kanren Deni (ERP) Manual-P300 wo Chushin ni (Event-Related Potential (ERP) Manual-Mainly on P300)”, Shinoharashinsha Publishers Inc., 1995, p. 30, the waveform of an event-related potential generally varies (shifts) from person to person by 30 ms to 50 ms. Thus, herein, the expression of the time component of “approximately X ms” or “around X ms” includes a range with a margin of 30 ms to 50 ms with respect to the time point X ms before and after the time point X ms. Examples of such a range are 100 ms±30 ms (in the case of X=100) and 200 ms±50 ms (in the case of X=200).

The term “task” refers to an assignment carried out by a user when the bioelectric potential is measured. Examples of the task include an assignment for pressing a button after five seconds from a start point set in advance and an assignment for taking an action in accordance with contents displayed on a screen.

### Underlying Knowledge of Inventors

As described in the “BACKGROUND” section, machine learning attracts attention as a method for highly accurately realizing complex classification and recognition that have hitherto been difficult. The inventors have studied machine learning that handles biological data items such as biosignals. Biosignals are analyzed to obtain the state of a user. As described above, the recognition apparatus disclosed in PTL 1 learns relationships between biological information of a user, such as a patient, and feeling information of the user in advance and recognizes a feeling of the user by using the result of learning and biological information of the user.

Increasing the recognition accuracy achieved by machine learning in such a recognition apparatus requires preparation of many biological data items. To collect biological data items, measurement experiments need to be performed on subjects. In the case where many biological data items are collected through measurement experiments, the measurement experiment for each subject takes long time, increasing the load imposed on the subject.

For example, a technique called data augmentation is a conventional technique for reducing a load of data preparation used hitherto in the field of machine learning. According to this data augmentation technique, pseudo data items are generated from an existing data item, and consequently an amount of data used in machine learning is increased. As described above, NPL 1 discloses a data argumentation technique used in an image recognition field. According to the technique of NPL 1, parametric deformations such as luminance adjustment, inversion, distortion, enlargement, and reduction are performed on an obtained image data item to generate pseudo training data items.

However, the conventional data augmentation techniques used in the image recognition field such as the one disclosed in NPL 1 are not suitable for data augmentation of biological data items such as biosignals because characteristics of natural images that are taken into account in data augmentation of image data items differ from characteristics that are to be taken into account in data augmentation of biological data items.

In data augmentation of image data items, for example, adjustment of luminance of an image simulates a change in a characteristic of brightness in a natural image. Enlargement or reduction of an image simulates a difference between images in terms of a characteristic of a distance between a target to be recognized and a camera. As described above, data augmentation techniques used for image data items are data augmentation techniques that take into account fluctuation factors of characteristics of an image. By generating image data items that reflect expected fluctuations of characteristics of an image using such a data augmentation technique in advance, an improved recognition rate is achieved in image recognition.

However, concepts such as the luminance and the size of a target are absent in characteristics of biological data items. Thus, it is necessary to find the characteristics fluctuation factors unique to biological data items, and a data augmentation technique that handles such characteristics fluctuation factors is needed for data augmentation of biological data items.

Thus, the inventors have considered a data generation apparatus and the like that implement data augmentation suitable for characteristics of biological data items. The inventors have further considered a classifier generation apparatus and the like that apply data augmentation to a small quantity of biological data items to generate enhanced training data items and that generate a classifier having a sufficient classification performance using the enhanced training data items. The inventors set a latency in a biological data item represented in time series and an amplitude of a bioelectric potential in the latency as characteristics of the biological data item and have conceived a data generation apparatus and the like based on the characteristics.

More specifically, a technique according to the present disclosure conceived by the inventors handles a biological data item relating to the occurrence of an event. An internal state of a subject is successfully estimated based on a change in a biological data item that starts from a timing at which the subject is stimulated by the occurrence of an event. For example, in the case where a subject is externally stimulated, a timing at which the subject is externally stimulated is identified clearly and easily. A change in a biological data item that starts from the timing at which the subject is stimulated is sometimes called an event-related potential in terms of brainwaves, for example. The inventors have considered that characteristics of a biological data item relating to the occurrence of an event are the latency and an amount of change in the bioelectric potential. The amount of change in the bioelectric potential is an amplitude of the potential and is an amount of change in magnitude of the potential relative to 0 V. Note that a fluctuation of the latency relates mainly to an internal factor of the subject and a fluctuation of the amplitude of the bioelectric potential relates mainly to an external factor of the subject.

The aforementioned characteristics will be described by using brainwave data as an example. Components representing an event-related potential include P300. P300 indicates a component having a peak at a potential greater than 0 V after 200 to 700 ms from the occurrence of an external stimulus or an internal stimulus. The character “P” indicates the positive component of the potential. It is considered that various components are mixed in P300. For example, it is known that the latency of the peak fluctuates from subject to subject for the same stimulus and, even for the same subject, the latency of the peak fluctuates every occurrence of an event such as every trial of a task. Such fluctuations are considered to directly influence the recognition accuracy of the pattern of P300. Thus, the latency of the peak relates to the internal factor of the user described above.

It is also considered that the amplitude fluctuates because of multiple factors. A possible internal factor is that the magnitude of the activity of the brain is not even and may fluctuate depending to the subject and the situation. A possible external factor may be a fluctuation of the measurement condition. Examples of the fluctuation of the measurement condition include a fluctuation that results from the state in which contact impedances between the scalp and electrodes are not constant in multiple brainwave measurements. In brainwave measurements, electrodes need to be in contact with the scalp. However, it is difficult to keep the contact impedances between the scalp and the electrodes constant in every measurement. As a result, the impedances fluctuate and thus the measured amplitude fluctuates. Specifically, the measured amplitude decreases as the contact impedance increases. The inventors have considered that these fluctuations also influence the recognition accuracy of the pattern.

Thus, the inventors have conceived to use the fluctuation of the latency of the peak and the fluctuation of the amplitude, which are characteristic fluctuation factors of biological data items, in a data augmentation technique in place of the parametric deformations such as adjustment of luminance and enlargement/reduction performed on image data items. This consequently enables generation of pseudo data items that reflect the ordinarily occurring fluctuations of biological data items in advance. Further, machine learning using such pseudo data items as training data items enables a classifier to have an improved accuracy. The inventors have conceived techniques described below based on the knowledge described above.

Specifically, a data generation apparatus according to one aspect of the present disclosure includes a memory that stores a generation rule including one or more time change values and/or one or more amplitude change values, and a processor that (a1) obtains a first biological data item including an event-related potential of a user and obtains a first label associated with the first biological data item, (a2) refers to the generation rule and generates one or more second biological data items by changing the first biological data item using the one or more time change values and/or the one or more amplitude change values, and (a3) outputs the one or more second biological data items associated with the first label, as training data related to the user.

In the above aspect, the fluctuation of the latency of the event-related potential relates to an internal factor of a user, and the fluctuation of the amplitude of the event-related potential relates to an external factor of the user. Biological data items generated using time change values simulate fluctuations of a biological data item due to the internal factor of the user. Biological data items generated using amplitude change values simulate fluctuations of the biological data item due to the external factor of the user. Since the one or more second biological data items including such biological data items have a label identical to the label of the first biological data item, the one or more second biological data items can serve as pseudo biological data items of the first biological data item. Thus, the data generation apparatus enables generation of pseudo biological data items from an existing biological data item and consequently enables generation of a large quantity of biological training data items.

In the data generation apparatus according to the one aspect of the present disclosure, the processor (a4), before the (a2), may obtain information on an event interval corresponding to the event-related potential and extract a first time interval included in the first biological data item with reference to the information on the event interval, and in the (a2), may generate the one or more second biological data items by changing the first biological data item in the first time interval using the one or more time change values and/or the one or more amplitude change values.

According to the above aspect, the one or more second biological data items can serve as pseudo biological data items of an event interval of the first biological data item. Thus, the second biological data items can virtually represent biological data items obtained when the event occurs. An example of the information on the event interval may be information on a start point of an event corresponding to the event interval.

In the data generation apparatus according to the one aspect of the present disclosure, the first biological data item may include an event-related potential of a brainwave of the user, the one or more time change values may be values for changing a measurement time of the first biological data item in a range from −250 ms to +250 ms, and the one or more amplitude change values may be values for changing an amplitude of the first biological data item in a range from 0.1 times to 1.9 times.

A biological data measurement system according to one aspect of the present disclosure includes the data generation apparatus, and a biological data measurer that is attached to the user and that measures the first biological data item from the user.

According to the above aspect, the biological data measurement system is capable of measuring the first biological data item from the user and generating the one or more second biological data items by using the measured first biological data item.

A classifier generation apparatus according to one aspect of the present disclosure includes a memory that stores a generation rule including one or more time change values and/or one or more amplitude change values, and a processor that (b1) obtains a first biological data item including an event-related potential of a user, a third biological data item including an event-related potential of the user, a first label associated with the first biological data item, and a second label associated with the third biological data item, (b2) refers to the generation rule and generates one or more second biological data items and one or more fourth biological data items by respectively changing the first biological data item and the third biological data item using the one or more time change values and/or the one or more amplitude change values, and (b3) generates a classifier using training data that includes the first biological data item, the one or more second biological data items, the third biological data item, and the one or more fourth biological data items.

According to the above aspect, the classifier generation apparatus is capable of generating pseudo biological data items having a corresponding label from an existing biological data item, just like the biological data generation apparatus. Further, the classifier generation apparatus is capable of generating a classifier by using many biological data items including the existing biological data item and the pseudo biological data items. Thus, the performance of the classifier can be improved. The classifier generated using biological data items with various labels is capable of classifying various labels from biological data items.

In the classifier generation apparatus according to the one aspect of the present disclosure, the processor (b4), before the (b2), may obtain information on event intervals corresponding to the respective event-related potentials and extract a first time interval and a third time interval included respectively in the first biological data item and the third biological data item with reference to the information on the event intervals, and in the (b2), may generate the one or more second biological data items and the one or more fourth biological data items by respectively changing the first biological data item in the first time interval and the third biological data item in the third time interval using the one or more time change values and/or the one or more amplitude change values.

According to the above aspect, the one or more second biological data items and the one or more fourth biological data items can virtually represent biological data items obtained when the events occur. Thus, a classifier capable of classifying such events can be generated.

In the classifier generation apparatus according to the one aspect of the present disclosure, each of the first biological data item and the third biological data item may include an event-related potential of a brainwave of the user, the one or more time change values may be values for changing a measurement time of each of the first biological data item and the third biological data item in a range from −250 ms to +250 ms, and the one or more amplitude change values may be values for changing an amplitude of each of the first biological data item and the third biological data item in a range from 0.1 times to 1.9 times.

A data generation method according to one aspect of the present disclosure includes (a1) obtaining a first biological data item including an event-related potential of a user and obtaining a first label associated with the first biological data item, (a2) referring to a generation rule that is stored in a memory and that includes one or more time change values and/or one or more amplitude change values and generating one or more second biological data items by changing the first biological data item using the one or more time change values and/or the one or more amplitude change values, and (a3) outputting the one or more second biological data items associated with the first label, as training data related to the user, at least one of the (a1), the (a2), and the (a3) being performed by a processor. According to the above aspect, substantially the same advantages as those of the data generation apparatus according to the one aspect of the present disclosure are obtained.

The data generation method according to the one aspect of the present disclosure may further include (a4), before the (a2), obtaining information on an event interval corresponding to the event-related potential and extracting a first time interval included in the first biological data item with reference to the information on the event interval, in which in the (a2), the one or more second biological data items may be generated by changing the first biological data item in the first time interval using the one or more time change values and/or the one or more amplitude change values.

In the data generation method according to the one aspect of the present disclosure, the first biological data item may include an event-related potential of a brainwave of the user, the one or more time change values may be values for changing a measurement time of the first biological data item in a range from −250 ms to +250 ms, and the one or more amplitude change values may be values for changing an amplitude of the first biological data item in a range from 0.1 times to 1.9 times.

A classifier generation method according to one aspect of the present disclosure includes (b1) obtaining a first biological data item including an event-related potential of a user, a third biological data item including an event-related potential of the user, a first label associated with the first biological data item, and a second label associated with the third biological data item, (b2) referring to a generation rule that is stored in a memory and that includes one or more time change values and/or one or more amplitude change values and generating one or more second biological data items and one or more fourth biological data items by respectively changing the first biological data item and the third biological data item using the one or more time change values and/or the one or more amplitude change values, and (b3) generating a classifier using training data that includes the first biological data item, the one or more second biological data items, the third biological data item, and the one or more fourth biological data items, at least one of the (b1), the (b2), and the (b3) being performed by a processor. According to the above aspect, substantially the same advantages as those of the classifier generation apparatus according to the one aspect of the present disclosure are obtained.

The classifier generation method according to the one aspect of the present disclosure may further include (b4), before the (b2), obtaining information on event intervals corresponding to the respective event-related potentials and extracting a first time interval and a third time interval respectively included in the first biological data item and the third biological data item with reference to the information on the event intervals, in which in the (b2), the one or more second biological data items and the one or more fourth biological data items may be generated by respectively changing the first biological data item in the first time interval and the third biological data item in the third time interval using the one or more time change values and/or the one or more amplitude change values.

In the classifier generation method according to the one aspect of the present disclosure, each of the first biological data item and the third biological data item may include an event-related potential of a brainwave of the user, the one or more time change values may be values for changing a measurement time of each of the first biological data item and the third biological data item in a range from −250 ms to +250 ms, and the one or more amplitude change values may be values for changing an amplitude of each of the first biological data item and the third biological data item in a range from 0.1 times to 1.9 times.

A recording medium according to one aspect of the present disclosure storing a control program causing a device including a processor to perform a process, the recording medium being non-volatile and computer-readable, the process including (a1) obtaining a first biological data item including an event-related potential of a user and obtaining a first label associated with the first biological data item, (a2) referring to a generation rule that is stored in a memory and that includes one or more time change values and/or one or more amplitude change values and generating one or more second biological data items by changing the first biological data item using the one or more time change values and/or the one or more amplitude change values, and (a3) outputting the one or more second biological data items associated with the first label, as training data related to the user. According to the above aspect, substantially the same advantages as those of the data generation apparatus according to the one aspect of the present disclosure are obtained.

In the recording medium according to the one aspect of the present disclosure, the process may further include (a4), before the (a2), obtaining information on an event interval corresponding to the event-related potential and extracting a first time interval included in the first biological data item with reference to the information on the event interval, in which in the (a2), the one or more second biological data items may be generated by changing the first biological data in the first time interval using the one or more time change values and/or the one or more amplitude change values.

In the recording medium according to the aspect of the present disclosure, the first biological data item may include an event-related potential of a brainwave of the user, the one or more time change values may be values for changing a measurement time of the first biological data item in a range from −250 ms to +250 ms, and the one or more amplitude change values may be values for changing an amplitude of the first biological data item in a range from 0.1 times to 1.9 times.

A recording medium according to another aspect of the present disclosure storing a control program causing a device including a processor to perform a process, the recording medium being non-volatile and computer-readable, the process including (b1) obtaining a first biological data item including an event-related potential of a user, a third biological data item including an event-related potential of the user, a first label associated with the first biological data item, and a second label associated with the third biological data item, (b2) referring to a generation rule that is stored in a memory and that includes one or more time change values and/or one or more amplitude change values and generating one or more second biological data items and one or more fourth biological data items by respectively changing the first biological data item and the third biological data item using the one or more time change values and/or the one or more amplitude change values, and (b3) generating a classifier using training data that includes the first biological data item, the one or more second biological data items, the third biological data item, and the one or more fourth biological data items. According to the above aspect, substantially the same advantages as those of the classifier generation apparatus according to the one aspect of the present disclosure are obtained.

In the recording medium according to the other aspect of the present disclosure, the process may further include (b4), before the (b2), obtaining information on event intervals corresponding to the respective event-related potentials and extracting a first time interval and a third time interval respectively included in the first biological data item and the third biological data item with reference to the information on the event intervals, in which in the (b2), the one or more second biological data items and the one or more fourth biological data items may be generated by respectively changing the first biological data item in the first time interval and the third biological data item in the third time interval using the one or more time change values and/or the one or more amplitude change values.

In the recording medium according to the other aspect of the present disclosure, each of the first biological data item and the third biological data item may include an event-related potential of a brainwave of the user, the one or more time change values may be values for changing a measurement time of each of the first biological data item and the third biological data item in a range from −250 ms to +250 ms, and the one or more amplitude change values may be values for changing an amplitude of each of the first biological data item and the third biological data item in a range from 0.1 times to 1.9 times.

It should be noted that general or specific embodiments may be implemented as a system, an apparatus, a method, an integrated circuit, a computer program, a computer-readable recording medium such as a recording disc, or any selective combination thereof. Examples of the computer-readable recording medium include a non-volatile recording medium, for example, a CD-ROM.

Embodiments of the present disclosure will be described specifically below with reference to the drawings. Note that each of the embodiments to be described below provides general or specific examples. The values, shapes, components, arrangement and connection of the components, steps (processes), the order of steps, etc., described in the following embodiments are merely illustrative and are not intended to limit the present disclosure. Among the components described in the following embodiments, a component not recited in any of the independent claims indicating the most generic concept is described as an optional component. In the following description of the embodiments, the expressions accompanying “substantially”, such as substantially parallel and substantially orthogonal, are sometimes used. For example, the expression “substantially parallel” not only indicates the state of being completely parallel but also indicates the state of being substantially parallel, that is, the state of allowing an error of several percent, for example. The same applies to other expressions accompanying “substantially”. In addition, each of the drawings is a schematic drawing and does not necessarily illustrate components strictly. Further, substantially the same components are denoted by the same reference signs in the drawings, and a duplicate description is sometimes omitted or simplified.

In embodiments below, an example of brainwave data items in an event of receiving a stimulus relating to a motivation state of a person will be described as an example of biological data items. Note that another biosignal such as an electrooculogram, an electrocardiogram, or an electromyogram may be used as the biological data items.

### First Embodiment

### 1-1. Configuration of Data Generation Apparatus

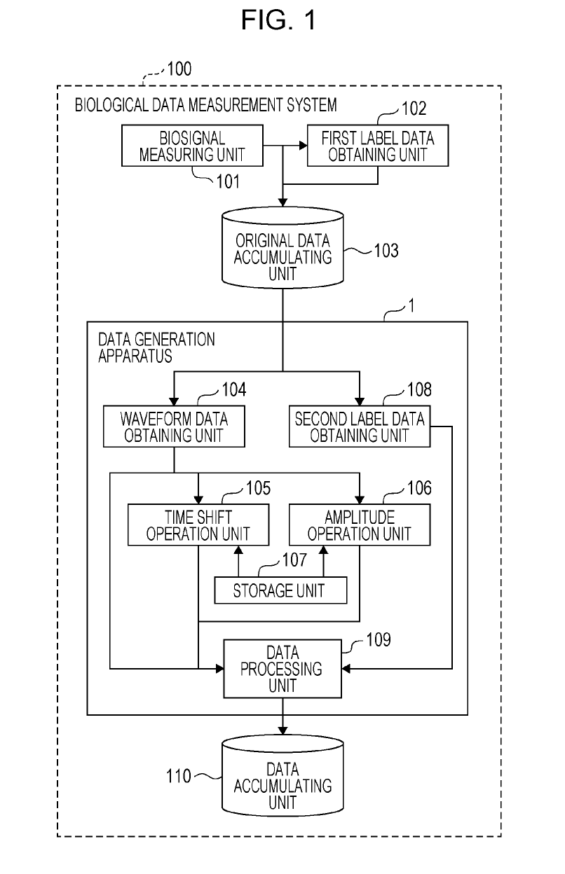

A configuration of a data generation apparatus 1 according to a first embodiment will be described. FIG. 1 illustrates a functional configuration of a biological data measurement system 100 including the data generation apparatus 1 according to the first embodiment. As illustrated in FIG. 1, the biological data measurement system 100 includes a biosignal measuring unit 101, a first label data obtaining unit 102, an original data accumulating unit 103, the data generation apparatus 1, and a data accumulating unit 110. In addition, the data generation apparatus 1 includes a waveform data obtaining unit 104, a time shift operation unit 105, an amplitude operation unit 106, a storage unit 107, a second label data obtaining unit 108, and a data processing unit 109.

### Biosignal Measuring Unit 101

The biosignal measuring unit 101 measures a biosignal when a user performs a task. An example of hardware of the biosignal measuring unit 101 is a sensor. An example of the sensor is an electroencephalograph that measures brainwave signals of a user. For example, the electroencephalograph is an electrometer that includes electrodes and measures potential differences across the electrodes.

The biosignal measuring unit 101 may measure a biosignal of the user all the time. In this case, part of the measured biosignal corresponds to a biosignal obtained when the user performs a task.

Alternatively, the biosignal measuring unit 101 may include a processor. The processor receives a timing at which the user performs a task and measures a biosignal at that timing using the sensor of the biosignal measuring unit 101. An external computer displays information for performing a task to the user using a display. Alternatively, an external computer presents information for performing a task to the user using a speaker. In such a case, the processor may receive the task start timing from the external computer.

A task performed by a user is an assignment performed by the user when a biosignal is measured and is an event performed to stimulate the user. The biosignal measuring unit 101 is an example of a biological data measuring unit.

Examples of the task include an assignment of pressing a button after five seconds from a start timing set in advance. In the first embodiment, a task relates to a motivation state of a person. A motivating task is an assignment that involves a user's active action. An example of the motivating task is an assignment in which the user looks at a clock and presses a button upon an elapse of five seconds from the start timing set in advance. A non-motivating task is an assignment that involves a user's passive action. An example of the non-motivating task is an assignment in which the user presses a button upon confirming that the clock stops after five seconds from the start timing set in advance. At that time, the action of the user relating to the task may be obtained using an interface device. An example of the interface device is a terminal including a button. The interface device obtains information indicating pressing of the button. Another example of the interface device is a mouse and keyboard used in a computer.

The biosignal measuring unit 101 also obtains information on a trigger indicating a timing at which the user performs the task at the same time. For example, if the task is the assignment of pressing a button as described above, the trigger is the timing, that is, the time at which the user presses the button. The biosignal measuring unit 101 obtains the information on the trigger from the interface device, for example. Alternatively, in the case where a timing at which the external computer outputs information for performing the task corresponds to the trigger, the biosignal measuring unit 101 obtains the information on the trigger from the external computer. The biosignal measuring unit 101 stores the measured biosignal and the trigger information in the original data accumulating unit 103 in association with each other. At that time, the biosignal measuring unit 101 may associate the biosignal with the measurement time of the biosignal and may store, in the original data accumulating unit 103, the biosignal and the measurement time as a biological data item including the biosignal and the measurement time.

In addition, a label is set for a task. The label indicates a type of the task. Alternatively, the label may indicate a state of a person relating to the type of the task. An example of the state of the person relating to the type of the task may be a psychological state of the person caused as a result of performing the task. For example, the biosignal measuring unit 101 may obtain, via the first label data obtaining unit 102, information on the label of the task that is performed. Then, the biosignal measuring unit 101 may associate the label with at least one of the biosignal and the measurement time and store them in the original data accumulating unit 103.

### First Label Data Obtaining Unit 102

The first label data obtaining unit 102 obtains a label of a task performed by a user when the biosignal measuring unit 101 measures a biosignal. The first label data obtaining unit 102 may obtain information on the label of the task via an interface device (not illustrated) included in the biological data measurement system 100. The information on the label of the task may be input to the interface device by the user or the like before the task is performed. The interface device may be a button, a switch, a key, a touch pad, or a microphone of a voice recognition device. Note that this interface device may be the same as the interface device that obtains the action of the user relating to the task. Further, the first label data obtaining unit 102 associates the obtained label with the biosignal measured by the biosignal measuring unit 101 and stores the label in the original data accumulating unit 103. Alternatively, the first label data obtaining unit 102 may obtain information including the biosignal and the trigger information from the biosignal measuring unit 101, obtain the label indicating the type of the task performed by the user from the obtained information, and store the label in the original data accumulating unit 103. In this case, the biosignal measuring unit 101 may obtain information on the label through an input from outside, for example.

For example, brainwave data items relating to the motivation state are measured in the first embodiment. In this case, the first label data obtaining unit 102 obtains one of two labels that are “motivating” and “non-motivating”. The first label data obtaining unit 102 associates such a label and the biosignal with each other and stores the label and the biosignal in the original data accumulating unit 103.

### Original Data Accumulating Unit 103

The original data accumulating unit 103 is capable of storing information and enables retrieval of the stored information. The original data accumulating unit 103 is implemented by a storage device, for example, a read-only memory (ROM), a random access memory (RAM), a semiconductor memory such as a flash memory, a hard disk drive, or a solid state drive (SSD). The original data accumulating unit 103 stores the biological data item obtained by the biosignal measuring unit 101 and data of the label obtained by the first label data obtaining unit 102. The biosignal measuring unit 101 may section the biological data item for each trial of the task on the basis of the trigger information to generate sectioned data items and may store the sectioned data items in the original data accumulating unit 103. The original data accumulating unit 103 stores the actually measured biological data item and the data of the label corresponding to the biological data item, that is, original data yet to be subjected to data augmentation. The processing of sectioning the biological data item for each trial of the task may be performed by the waveform data obtaining unit 104 (described later).

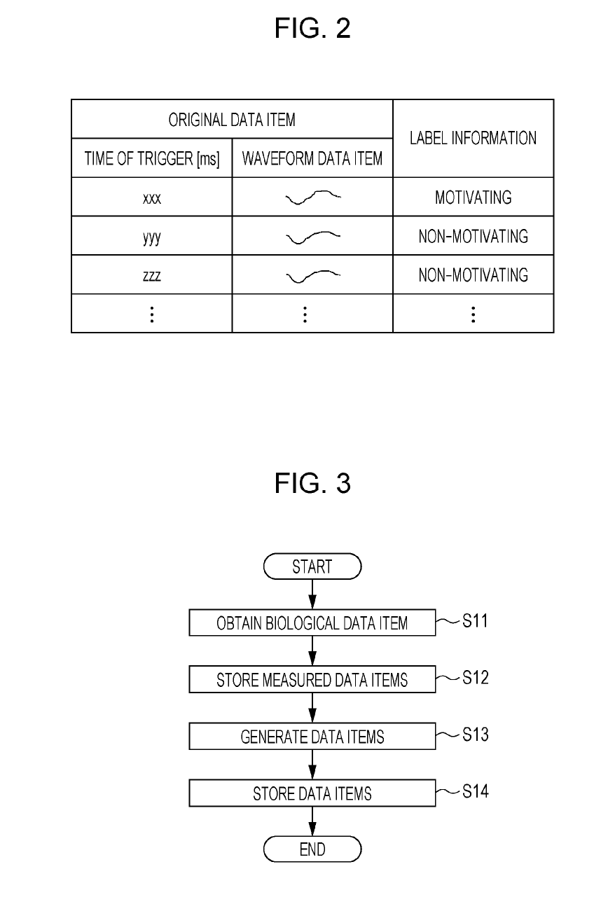

For example, FIG. 2 illustrates an example of the information stored in the original data accumulating unit 103. A biological data item that is illustrated as an original data item in FIG. 2 and label information corresponding to the biological data item are stored in the original data accumulating unit 103 in association with each other. The biological data item includes a group of biosignals and measurement times of the biosignals, which are represented as a waveform data item, and the time of the trigger. The group of biosignals, the measurement times, and the time of the trigger are stored in the original data accumulating unit 103 in association with one another. The group of biosignals is a set of biosignals within a single event interval and forms a waveform data item when being arranged in time series. A single event interval corresponds to a period of a single task performed for a single trigger, details of which will be described later. In the example of FIG. 2, the group of biosignals is represented as a waveform data item for ease of understanding. FIG. 2 illustrates a data item obtained when a task of pressing a button is performed. Thus, the time of the trigger indicates the timing at which the user presses the button, and the label information includes two labels of “motivating” and “non-motivating”.

### Waveform Data Obtaining Unit 104

The waveform data obtaining unit 104 obtains the biological data item and the trigger information from the original data accumulating unit 103. The waveform data obtaining unit 104 further generates a waveform data item of the biosignal from the biological data item and the trigger information. An example of the waveform data item of the biosignal is a waveform data item of the bioelectric potential, which is a brainwave waveform data item in the first embodiment. The waveform data item represents a change in potential against a time axis. The waveform data obtaining unit 104 outputs the generated waveform data item to the time shift operation unit 105, the amplitude operation unit 106, and the data processing unit 109.

### Time Shift Operation Unit 105

The time shift operation unit 105 obtains the waveform data item from the waveform data obtaining unit 104. The time shift operation unit 105 further generates (a) new waveform data item(s) by shifting the entire waveform of the obtained waveform data item by a time change value set in advance in a positive direction and/or a negative direction with respect to the time axis. In other words, a new waveform data item is generated by changing measurement time of the obtained waveform data item on the basis of a time change value. The time shift operation unit 105 performs the above processing in accordance with a generation rule stored in the storage unit 107 (described later). The new waveform data item is a pseudo waveform data item of the waveform data item obtained from the waveform data obtaining unit 104. By preparing multiple time change values, that is, multiple shift amounts, the quantity of generated waveform data items can be increased. Note that the time shift operation unit 105 may shift a portion of the obtained waveform data item. In addition, the time shift operation unit 105 may shift the waveform data item over the entire measurement period of the waveform data item or may shift the waveform data item over a portion of the measurement period. The time shift operation unit 105 performs data augmentation on the waveform data item including the measurement data in terms of time. The time shift operation unit 105 outputs the generated waveform data item to the data processing unit 109.

### Amplitude Operation Unit 106

The amplitude operation unit 106 obtains the waveform data from the waveform data obtaining unit 104. The amplitude operation unit 106 further generates a new waveform data item by changing the amplitude of the obtained waveform data item, specifically, by multiplying the amplitude of the obtained waveform data item by an amplitude change value set in advance. The amplitude operation unit 106 performs the above processing in accordance with a generation rule stored in the storage unit 107 (described later). The new waveform data item is a pseudo waveform data item of the waveform data item obtained from the waveform data obtaining unit 104. By preparing multiple amplitude change values, the quantity of generated waveform data items can be increased. Note that the amplitude operation unit 106 may change the amplitude of the entire waveform of the obtained waveform data item or the amplitude of a portion of the waveform data item. In addition, the amplitude operation unit 106 may change the amplitude of the waveform data item over the entire measurement period of the waveform data item or may change the amplitude of the waveform data item over a portion of the measurement period. The amplitude operation unit 106 performs data augmentation on the waveform data item including the measurement data in terms of amplitude. The amplitude operation unit 106 outputs the generated waveform data item to the data processing unit 109.

### Storage Unit 107

The storage unit 107 is capable of storing information and enables retrieval of the stored information. The storage unit 107 is implemented by a storage device, for example, a ROM, a RAM, a semiconductor memory such as a flash memory, a hard disk drive, or an SSD. The storage unit 107 stores the generation rules that are rules used when the time shift operation unit 105 and the amplitude operation unit 106 generate waveform data items through data augmentation. The generation rules include time change values for shifting a waveform data item and amplitude change values by which the amplitude of a waveform data item is multiplied. In addition, the generation rules may include a relationship between a quantity of new pseudo waveform data items generated by the time shift operation unit 105 from a single waveform data item and time change values and a relationship between a quantity of new pseudo waveform data items generated by the amplitude operation unit 106 and amplitude change values. In this case, once the quantity of new pseudo waveform data items is determined by the generation rules, the time change values or the amplitude change values can be automatically determined.

### Second Label Data Obtaining Unit 108

The second label data obtaining unit 108 obtains a label of a task corresponding to an original waveform data item yet to be subjected to data augmentation processing from the original data accumulating unit 103. The original waveform data item yet to be subjected to data augmentation processing is a waveform data item generated by the waveform data obtaining unit 104. Thus, the obtained label corresponds to the generation data obtained by the waveform data obtaining unit 104. The second label data obtaining unit 108 outputs information on the obtained label to the data processing unit 109. At that time, the second label data obtaining unit 108 may output, to the data processing unit 109, information that associates the biological data item constituting the waveform data item generated by the waveform data obtaining unit 104 with the label.

### Data Processing Unit 109

The data processing unit 109 integrates each of the pseudo waveform data item obtained from the time shift operation unit 105 and the pseudo waveform data item obtained from the amplitude operation unit 106 and the label information obtained from the second label data obtaining unit 108 together. The data processing unit 109 further integrates the waveform data item of the biological data item obtained from the waveform data obtaining unit 104 and the label information obtained from the second label data obtaining unit 108 together. Consequently, each waveform data item is labeled. The data processing unit 109 stores each waveform data item integrated with the label in the data accumulating unit 110. Thus, a waveform data item of a biological data item, a pseudo waveform data item obtained in terms of time, and a pseudo waveform data item obtained in terms of amplitude can be stored for the same label in the data accumulating unit 110.

### Data Accumulating Unit 110

The data accumulating unit 110 is capable of storing information and enables retrieval of the stored information. The data accumulating unit 110 is implemented by a storage device mentioned in the description of the original data accumulating unit 103. The data accumulating unit 110 stores data items output from the data processing unit 109. The data items stored in the data accumulating unit 110 are used by a classification apparatus 2 (described later). Specifically, the data items are used by a classifier of the classification apparatus 2 as training data for learning and are used as test data for measuring the performance of the classifier. The usage of the data items will be described later. Note that the classifier is also referred to as a classification model.

Note that the data generation apparatus 1 may obtain the biological data item and the label information without using the original data accumulating unit 103. This configuration enables real-time data augmentation processing in the data generation apparatus 1.

The first label data obtaining unit 102 and the components of the data generation apparatus 1, which are the waveform data obtaining unit 104, the time shift operation unit 105, the amplitude operation unit 106, the second label data obtaining unit 108, and the data processing unit 109, may be constituted by a computer system (not illustrated) including a processor such as a central processing unit (CPU), a RAM, and a ROM. Functions of some or all of the components mentioned above may be achieved as a result of the CPU executing a program stored in the ROM by using the RAM as a work memory. In addition, functions of some or all of the components mentioned above may be achieved by a dedicated hardware circuit, such as an electronic circuit or an integrated circuit. The program may be stored in the ROM in advance or may be provided as an application via communication through a communication network such as the Internet, via communication based on a mobile communication standard, via a wireless network of another kind, via a wired network, via broadcasting, etc.

The biological data measurement system 100 may be constituted by a single apparatus or by multiple individual apparatuses. The data generation apparatus 1 and at least one of the biosignal measuring unit 101, the first label data obtaining unit 102, and the original data accumulating unit 103 may constitute a single apparatus, or the data generation apparatus 1 may be separated from any of the biosignal measuring unit 101, the first label data obtaining unit 102, and the original data accumulating unit 103. The data generation apparatus 1 and the data accumulating unit 110 may constitute a single apparatus, or the data generation apparatus 1 may be separated from the data accumulating unit 110. In the case where the data generation apparatus 1 is separated from the other components of the biological data measurement system 100, the data generation apparatus 1 may be connected to the components via wired or wireless communication. The wireless communication may be wireless communication via a communication network such as the Internet. Examples of such wireless communication include a wireless local area network (LAN) such as Wireless Fidelity (Wi-Fi (registered trademark)).

### 1-2. Operation of Data Generation Apparatus

An operation performed by the biological data measurement system 100 will be described next mainly on an operation performed by the data generation apparatus 1 according to the first embodiment. FIG. 3 is a flowchart illustrating an example of a flow of the operation performed by the biological data measurement system 100.

### Step S11

As illustrated in FIG. 3, the biosignal measuring unit 101 measures and obtains a brainwave data item of a user to whom the biosignal measuring unit 101 is attached. The brainwave data item is a data item represented in time series and is a biological data item. The biosignal measuring unit 101 also obtains trigger information indicating a timing at which the user performs a task. For example, in the case where the task is an assignment of pressing a button after five seconds from a start timing set in advance, the biosignal measuring unit 101 obtains a signal generated by the button upon the user pressing of the button. The biosignal measuring unit 101 sets the time point at which it obtains the signal as trigger occurrence timing. As described above, the biosignal measuring unit 101 obtains trigger information by obtaining a signal that is input when a user performs a task. Note that the trigger occurrence timing may be identified by a device other than the biosignal measuring unit 101, and the biosignal measuring unit 101 may obtain information on the trigger occurrence timing from the device. The biosignal measuring unit 101 attaches the trigger information to the biological data item.

### Step S12

Then, the biosignal measuring unit 101 sections the biological data item obtained in step S11 for predetermined intervals and stores the sectioned biological data items in the original data accumulating unit 103 in step S12. When sectioning the biological data item for the predetermined intervals, the biosignal measuring unit 101 uses the trigger information obtained in step S11. Specifically, the biosignal measuring unit 101 sets the timing at which the trigger occurs, which is the trigger occurrence timing, as the start point of a biological data item of a predetermined interval. The predetermined interval is an interval corresponding to a period of one trial of a task. Hereinafter, the predetermined interval is also referred to as an event interval. The event interval is a period including one trial of a task and is a period in which an event called a task occurs once.

For example, in the case where the user tries a task multiple times and there are multiple trigger occurrence timings, the event interval may be a period between the trigger occurrence timings. Alternatively, the event interval may be a period from when a trigger occurs to when a fluctuation of the biological data item resulting from the task converges. Note that the biological data item sectioned for the event interval may include a biological data item obtained before the trigger occurrence timing. Such a biological data item can represent a change before and after the occurrence of the trigger. In addition, the biosignal measuring unit 101 performs, as preprocessing of sectioning the biological data item for event intervals, on the biological data item, noise removal using a bandpass filter, baseband correction, and removal of trials of the task including outliers that are values greater than or equal to a given threshold set in advance. Note that a range of a biosignal, specifically, a range of the amplitude of a brainwave signal is assumed in advance. The threshold is set for this amplitude range, and values greater than or equal to this threshold are considered to be outliers. Then, trials of a task including measurement results of the brainwave signal having a value greater than or equal to the threshold are removed from the target that is sectioned for the event intervals.

In addition, the first label data obtaining unit 102 obtains a label indicating the type of the task performed when the biological data item is measured, from the biological data item obtained by the biosignal measuring unit 101 in step S11. The first label data obtaining unit 102 associates the label information with the sectioned biological data item of the event interval obtained by the biosignal measuring unit 101 and stores the label information and the sectioned biological data item in the original data accumulating unit 103. Note that the first label data obtaining unit 102 may obtain the label information via an input to an input device (not illustrated) of the biological data measurement system 100.

The sectioned biological data item of the event interval obtained by the biosignal measuring unit 101 is data representing a temporal change in bioelectric potential starting from the trigger occurrence timing. Such a biological data item of the event interval represents a user's temporal electrophysiological response in response to a stimulus resulting from an objectively defined event of pressing a button and corresponds to an event-related potential. Examples of the stimulus include a visual stimulus or an auditory stimulus. The event-related potential can be represented by a temporal change in potential. For example, in the case of measuring a brainwave data item, the biosignal measuring unit 101 can output the bioelectric potential of the brain as the brainwave data item, that is, the event-related potential by detecting a periodical current caused by electrical activities of neurons of the brain. The event-related potential represents a temporal change in bioelectric potential of the brain. Such an event-related potential may be assumed to be brainwaves excluding bioelectric potentials of α, β, γ, θ, and δ waves.

### Step S13

Then, the waveform data obtaining unit 104 obtains the biological data item from the original data accumulating unit 103 and generates a waveform data item of a bioelectric potential, that is, a brainwave, from the obtained biological data item in step S13. The waveform data obtaining unit 104 outputs the generated waveform data item to the data processing unit 109 and at least one of the time shift operation unit 105 and the amplitude operation unit 106. The time shift operation unit 105 generates pseudo waveform data items, which are new waveform data items, by shifting the obtained waveform data item by a time change value set in advance in a positive direction and/or a negative direction with respect to the time axis. The amplitude operation unit 106 generates pseudo waveform data items, which is new waveform data items, by changing the amplitude of the obtained waveform data item, specifically, by multiplying the amplitude of the obtained waveform data item by an amplitude change value set in advance. At that time, the time shift operation unit 105 and the amplitude operation unit 106 perform the above-described processing in accordance with the generation rules stored in the storage unit 107. Consequently, the waveform data item generated from the biological data item and at least two pseudo waveform data items generated by shifting of time and by changing of the amplitude are generated. Thus, the amount of waveform data items increases and data augmentation of the waveform data items is performed.

In addition, the second label data obtaining unit 108 obtains a label corresponding to the generation data obtained by the waveform data obtaining unit 104 from the original data accumulating unit 103 and outputs the label information to the data processing unit 109.

### Step S14

Then, the data processing unit 109 integrates the label information obtained from the second label data obtaining unit 108 and each of the waveform data item of the biological data item obtained from the waveform data obtaining unit 104, the pseudo waveform data item obtained from the time shift operation unit 105, and the pseudo waveform data item obtained from the amplitude operation unit 106 together. That is, the data processing unit 109 associates the label with the waveform data item generated from the biological data item, the pseudo waveform data item generated by shifting of time, and the pseudo waveform data item generated by changing of the amplitude. The data processing unit 109 stores the waveform data items integrated with the label information in the data accumulating unit 110.

As a result of the processing of steps S11 to S14 described above, data augmentation is performed on the biological data item which is a measurement data item, and consequently data including the actually measured data item and pseudo data items based on the actually measured data item is generated. Such data augmentation can increase the amount of data by several to several tens of times.

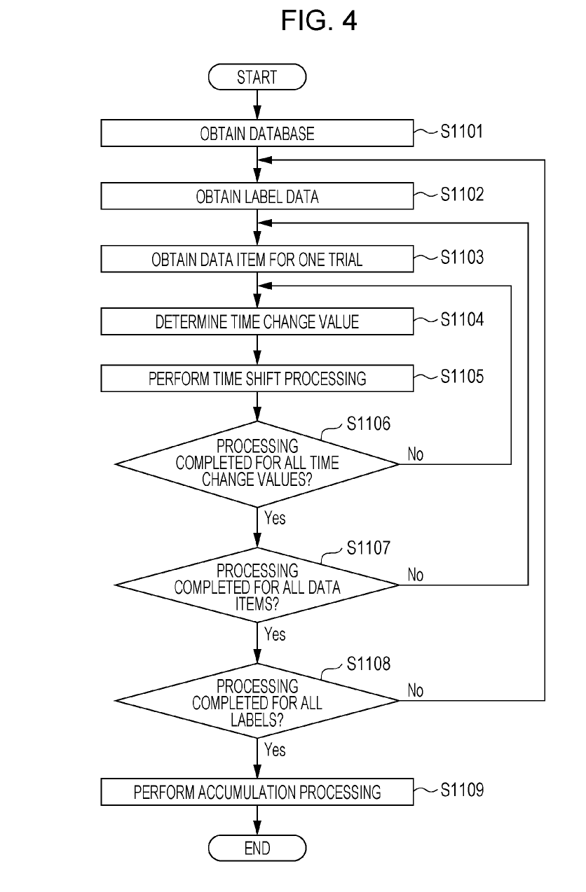

Details of the operation performed by the time shift operation unit 105 will be further described with reference to FIG. 4. FIG. 4 is a flowchart illustrating an example of a flow of the operation performed by the time shift operation unit 105 of the data generation apparatus 1 according to the first embodiment.

In step S1101, the waveform data obtaining unit 104 obtains biological data items, that is, a database, from the original data accumulating unit 103 and generates waveform data items. The biological data items include biological data items corresponding to different labels. Further, a biological data item corresponding to one label includes results of multiple trials of a task. Thus, the waveform data obtaining unit 104 generates multiple waveform data items corresponding to the respective trials.

In step S1102, the second label data obtaining unit 108 obtains label information corresponding to the biological data items obtained by the waveform data obtaining unit 104. The second label data obtaining unit 108 further selects one label from among labels included in the label information. This selected label is a label for which processing of steps S1103 to S1107 (described later) is not performed yet.

In step S1103, the time shift operation unit 105 obtains information on the selected one label from the second label data obtaining unit 108. The time shift operation unit 105 further obtains a waveform data item for one trial of the task corresponding to the obtained label from the waveform data items generated by the waveform data obtaining unit 104. This waveform data item corresponds to a waveform data item of an event interval. At that time, the time shift operation unit 105 selects a waveform data item for which the processing of steps S1104 to S1106 (described later) is not performed yet from among the waveform data items corresponding to the obtained one label.

In step S1104, the time shift operation unit 105 determines parameters for data augmentation processing. Specifically, the time shift operation unit 105 refers to the generation rule stored in the storage unit 107 and determines a time change value used for the waveform data item and the quantity of new waveform data items to be generated based on the time change value in accordance with this generation rule. Note that the time change value may be in a range from−250 ms to +250 ms or in a range from −50 ms to +50 ms.

For example, the time shift operation unit 105 performs processing in accordance with a table such as Table 1 below. Table 1 shows the quantity of waveform data items after data augmentation as a multiplying factor relative to the quantity of waveform data items before data augmentation. In Table 1, multiplying factors of ×1 to ×11 are set. Further, for each of the multiplying factors, the time change values are set. Time change values “±n ms” indicate that two waveform data items are generated by shifting a waveform data item by “+n ms” and “−n ms” along the time axis. The time shift operation unit 105 determines the multiplying factor, that is, an increasing rate, of the amount of data in accordance with an instruction received from a user or the like and obtains time change values by searching the table such as Table 1 for the determined multiplying factor. Then, the time shift operation unit 105 selects one time change value that is not used in processing of step S1105 (described later) yet from among the time change values. Note that a given value is selected from values in a range greater than 0 and less than or equal to 50 and is determined as the value of “n”. For example, the value of “n” can be determined such that the time change value is within the range for each multiplying factor. Thus, the quantity for the value of “n” may be different for different multiplying factors. In addition, the value of “n” may be selected from multiples of 5 in the range mentioned above. The value of “n” may be determined in advance or may be determined via an interface device. Table 1 shows merely an example of the generation rule, and the generation rule is not limited to this example.

TABLE 1
Relationship between Time Change
Values and Data Increasing Rate

| | | Time Change Values (Unit: ms) | | | | | |
|---|---|---|---|---|---|---|---|
| Data | ×1 | 0 | | | | | |
| Increasing | ×3 | 0 | ±n | | | | |
| Rate | ×5 | 0 | ±n | ±2n | | | |
| | ×7 | 0 | ±n | ±2n | ±3n | | |
| | ×9 | 0 | ±n | ±2n | ±3n | ±4n | |
| | ×11 | 0 | ±n | ±2n | ±3n | ±4n | ±5n |

In step S1105, the time shift operation unit 105 generates a new waveform data item by shifting the waveform data item obtained from the waveform data obtaining unit 104 based on the time change value determined in step S1104.

In step S1106, the time shift operation unit 105 determines whether the processing of step S1105 has been performed for all the time change values obtained in step S1104. If the time shift operation unit 105 determines that the processing of step S1105 has been performed for all the time change values (Yes in step S1106), the process proceeds to step S1107. If the time shift operation unit 105 determines that the processing of step S1105 has not been performed for all the time change values (No in step S1106), the process returns to step S1104.

As a result of repeatedly performing the processing of steps S1104 to S1106, multiple new waveform data items corresponding to respective time change values are generated for a single waveform data item. For example, when determining the multiplying factor to be “×11” in step S1104, the time shift operation unit 105 generates ten new waveform data items by shifting the waveform data item obtained from the waveform data obtaining unit 104 by “±n ms”, “±2n ms”, “±3n ms”, “±4n ms”, and “±5n ms” along the time axis. These new waveform data items are waveform data items generated by shifting a waveform data item obtained from the waveform data obtaining unit 104, for example, by “n ms” in the positive direction and the negative direction.

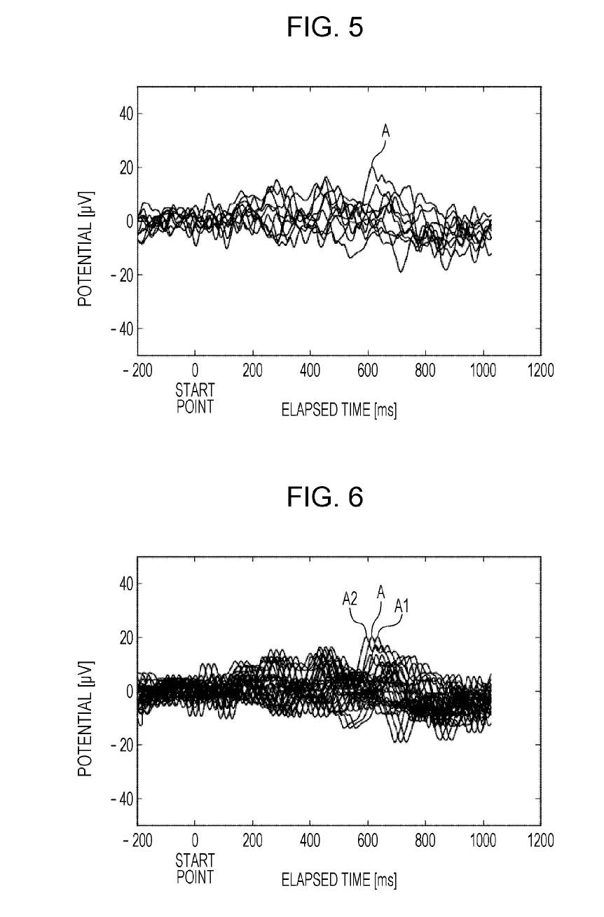

FIG. 5 illustrates an example of waveform data items of brainwaves generated by the waveform data obtaining unit 104. FIG. 6 illustrates an example of waveform data items generated by the time shift operation unit 105. Referring to FIG. 5, a time point corresponding to an elapsed time of “0 ms” is the trigger occurrence timing and is the start point of an event interval. The event interval corresponds to a period of one trial of a task and is a period from the elapsed time of −200 to the elapsed time of 1000 ms in this example. The example illustrated in FIG. 6 indicates the case where the time shift operation unit 105 determines the multiplying factor to be “×3” and the time change values to be “±n ms” in the table such as Table 1. The time shift operation unit 105 generates new waveform data items by shifting all the waveform data items illustrated in FIG. 5 by “±n ms”. For example, the time shift operation unit 105 shifts the entire waveform of a waveform data item A in the event interval in FIG. 5 by “+n ms” along the time axis that corresponds to the horizontal axes of FIGS. 5 and 6, that is, advances the entire waveform by “+n ms” to generate a waveform data item A1 illustrated in FIG. 6. In addition, the time shift operation unit 105 shifts the entire waveform of the waveform data A by “−n ms” along the time axis, that is, advances the entire waveform by “−n ms” to generate a waveform data item A2 illustrated in FIG. 6. Note that n=20 in the example of FIG. 6.

As a result of shifting the waveform data item A in the positive direction or the negative direction along the time axis as described above, part of the waveform data item A around the end or the start of the event interval moves to be outside the event interval. The start is the elapsed time of −200 ms and the end is the elapsed time of 1000 ms. In such a case, part of the waveform data item A that is moved to be outside the event interval around the end of the event interval is inserted at the start of the event interval in the waveform data item Al. In addition, part of the waveform data item A that is moved to be outside the event interval around the start of the event interval is inserted at the end of the event interval in the waveform data item A2. In this way, new waveform data items can be generated without changing the frequency component of the entire waveform data in the event interval.

In step S1107, the time shift operation unit 105 determines whether the processing of steps S1104 to S1106 has been performed for all the waveform data items corresponding to the label determined by the second label data obtaining unit 108 in step S1102. If the time shift operation unit 105 determines that the processing of steps S1104 to S1106 has been performed for all the waveform data items (Yes in step S1107), the process proceeds to step S1108. If the time shift operation unit 105 determines that the processing of steps S1104 to S1106 has not been performed for all the waveform data items (No in step S1107), the process returns to step S1103.

In step S1108, the time shift operation unit 105 determines whether the processing of steps S1103 to S1107 has been performed for all the labels included in the label information obtained by the second label data obtaining unit 108 in step S1102. If the time shift operation unit 105 determines that the processing of steps S1103 to S1107 has been performed for all the labels (Yes in step S1108), the process proceeds to step S1109. If the time shift operation unit 105 determines that the processing of steps S1103 to S1107 has not been performed for all the labels (No in step S1108), the process returns to step S1102.

In step S1109, the data processing unit 109 obtains the new waveform data items from the time shift operation unit 105 and obtains the label information from the second label data obtaining unit 108. The data processing unit 109 further associates the label information with each of the obtained new waveform data items to integrate them. The data processing unit 109 stores the waveform data items integrated with the label information in the data accumulating unit 110.

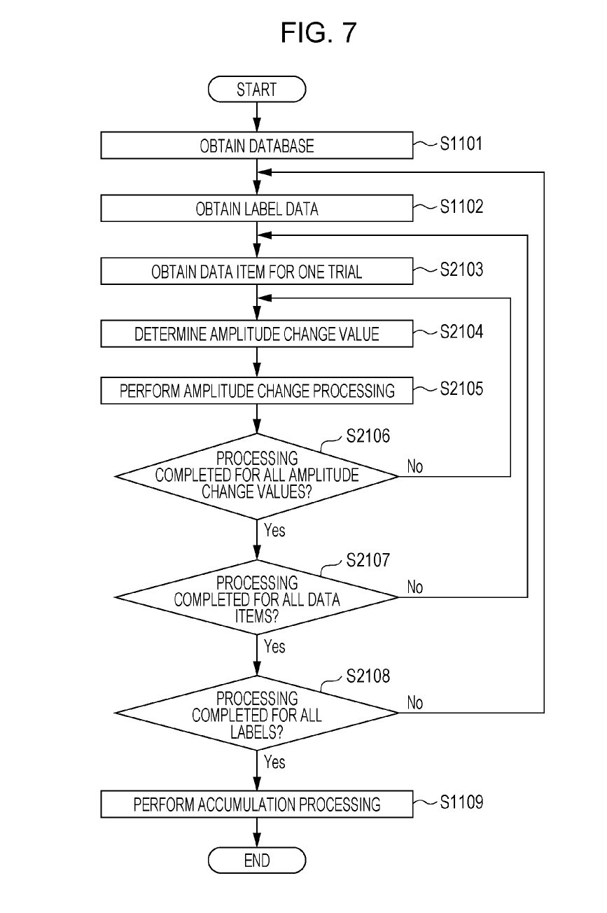

Details of the operation performed by the amplitude operation unit 106 will be described with reference to FIG. 7. FIG. 7 is a flowchart illustrating an example of a flow of the operation performed by the amplitude operation unit 106 of the data generation apparatus 1 according to the first embodiment.

First, as in the example of the time shift operation unit 105 illustrated in FIG. 4, processing of steps S1101 and S1102 is performed.

As in the processing performed by the time shift operation unit 105 in step S1103 of FIG. 4, the amplitude operation unit 106 obtains information on one label determined by the second label data obtaining unit 108 in step S2103. The amplitude operation unit 106 further obtains a waveform data item for one trial of a task corresponding to the obtained label. At that time, the amplitude operation unit 106 selects a waveform data item for which processing of steps S2104 to S2106 (described later) is not performed yet from among waveform data items corresponding to the obtained one label.

In step S2104, the amplitude operation unit 106 determines parameters for data augmentation processing. Specifically, the amplitude operation unit 106 refers to the generation rule stored in the storage unit 107 and determines amplitude change values used for the waveform data item and the quantity of new waveform data items generated by changing of the amplitude in accordance with this generation rule. Note that the amplitude change value may be a multiplying factor in a range from 0.1 times to 1.9 times or a multiplying factor in a range from 0.5 times to 1.5 times. The amplitude change value may be determined so as to satisfy the above range.

For example, the amplitude operation unit 106 performs processing in accordance with a table such as Table 2 below. Table 2 shows multiplying factors of the quantity of the waveform data items after data augmentation relative to the quantity of the waveform data items before data augmentation. Further, for each of the multiplying factors, amplitude change values are set. The amplitude change values “1±n/100 times” indicate that two waveform data items are generated by multiplying the amplitude of one waveform data item by “1+n/100 times” and “1−n/100 times”. The amplitude operation unit 106 determines the multiplying factor of the amount of data, that is, an increasing rate, in accordance with an instruction received from a user or the like and searches a table such as Table 2 for the determined multiplying factor to obtain multiple amplitude change values. Then, the amplitude operation unit 106 determines one amplitude change value that is not used in processing of step S2105 (described later) yet from among the multiple amplitude change values. Note that a given value is selected in a range exceeding 0 and less than or equal to 50 and is determined as the value of “n”. For example, the value of “n” can be determined such that the amplitude change value is within the above range for each multiplying factor. Thus, the quantity corresponding to the value of “n” may be different for different multiplying factors. In addition, the value of “n” may be selected from among multiples of 5 within the range mentioned above. The value of “n” may be determined in advance or may be determined via an interface device or the like. Table 2 shows merely an example of the generation rule, and the generation rule is not limited to this example.

TABLE 2
Relationship between Amplitude Change Values and Data Increasing Rate

| | | Amplitude Change Values (Unit: times) | | | | | |
|---|---|---|---|---|---|---|---|
| Data | ×1 | 1 | | | | | |
| Increasing | ×3 | 1 | 1 ± n/100 | | | | |
| Rate | ×5 | 1 | 1 ± n/100 | 1 ± 2n/100 | | | |
| | ×7 | 1 | 1 ± n/100 | 1 ± 2n/100 | 1 ± 3n/100 | | |
| | ×9 | 1 | 1 ± n/100 | 1 ± 2n/100 | 1 ± 3n/100 | 1 ± 4n/100 | |
| | ×11 | 1 | 1 ± n/100 | 1 ± 2n/100 | 1 ± 3n/100 | 1 ± 4n/100 | 1 ± 5n/100 |

In step S2105, the amplitude operation unit 106 generates a new waveform data item by multiplying the amplitude of the waveform data item obtained from the waveform data obtaining unit 104 based on the amplitude change value determined in step S2104.

In step S2106, the amplitude operation unit 106 determines whether the processing of step S2105 has been performed for all the amplitude change values obtained in step S2104. If the amplitude operation unit 106 determines that the processing of step S2105 has been performed for all the amplitude change values (Yes in step S2106), the process proceeds to step S2107. If the amplitude operation unit 106 determines that the processing of step S2105 has not been performed for all the amplitude change values (No in step S2106), the process returns to step S2104.

As a result of repeatedly performing the processing of steps S2104 to S2106, new waveform data items corresponding to the respective amplitude change values are generated for a waveform data item. For example, in the case of determining the multiplying factor to be “×7” in step S2104, the amplitude operation unit 106 generates six new waveform data items by multiplying the amplitude of the waveform data item obtained from the waveform data obtaining unit 104 by “1±n/100 times”, “1±2n/100 times”, and “1±3n/100 times”.

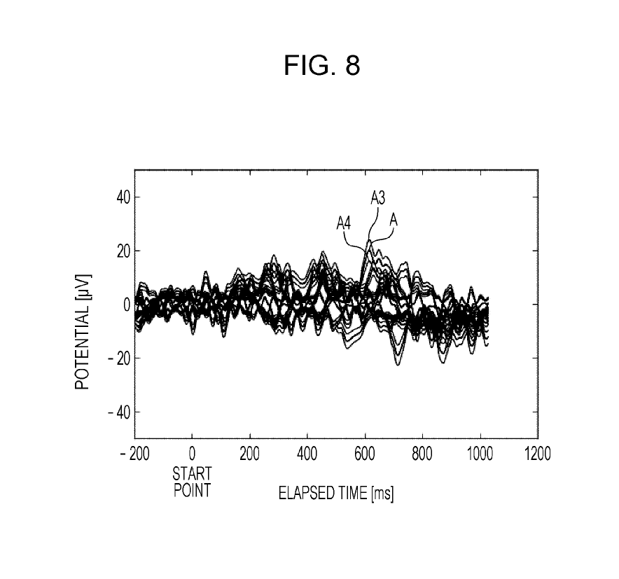

FIG. 8 illustrates an example of waveform data items generated by the amplitude operation unit 106 from the waveform data items illustrated in FIG. 5. The example illustrated in FIG. 8 shows the case where the amplitude operation unit 106 determines the multiplying factor to be “×3” and the amplitude change values to be “1±n/100 times” in the table such as Table 2. The amplitude operation unit 106 generates new waveform data items by multiplying the amplitudes of all the waveform data items illustrated in FIG. 5 by “1±n/100 times”. For example, the amplitude operation unit 106 generates a waveform data item A3 illustrated in FIG. 8 by multiplying the amplitude of the entire waveform of the waveform data item A within the event interval illustrated in FIG. 5 by “1+n/100 times”. The amplitude operation unit 106 also generates a waveform data item A4 illustrated in FIG. 8 by multiplying the amplitude of the entire waveform of the waveform data item A by “1−n/100 times”. Note that n=20 in FIG. 8. Multiplying the amplitude by “1+n/100 times” is multiplying for enlargement and is also called multiplying in a positive direction of the amplitude axis. Multiplying the amplitude by “1−n/100 times” is multiplying for reduction and is also called multiplying in a negative direction of the amplitude axis. The amplitude multiplying processing described above does not change the temporal position of the peak of the waveform in the waveform data item within the event interval before and after the multiplying. Thus, new waveform data items can be generated without changing the temporal peak position of the waveform data item in the event interval.

In step S2107, the amplitude operation unit 106 determines whether the processing of steps S2104 to S2106 has been performed for all the waveform data items corresponding to the label determined by the second label data obtaining unit 108 in step S1102. If the amplitude operation unit 106 determines that the processing of steps S2104 to S2106 has been performed for all the waveform data items (Yes in step S2107), the process proceeds to step S2108. If the amplitude operation unit 106 determines that the processing of steps S2104 to S2106 has not been performed for all the waveform data items (No in step S2107), the process returns to step S2103.

In step S2108, the amplitude operation unit 106 determines whether the processing of steps S2103 to S2107 has been performed for all the labels included in the label information obtained by the second label data obtaining unit 108 in step S1102. If the amplitude operation unit 106 determines that the processing of steps S2103 to S2107 has been performed for all the labels (Yes in step S2108), the process proceeds to step S1109. If the amplitude operation unit 106 determines that the processing of steps S2103 to S2107 has not been performed for all the labels (No in step S2108), the process returns to step S1102.

In step S1109, the data processing unit 109 obtains the new waveform data items from the amplitude operation unit 106 and obtains the label information from the second label data obtaining unit 108. The data processing unit 109 further associates the obtained new waveform data items with the label information to integrate them. The data processing unit 109 stores the waveform data items integrated with the label information in the data accumulating unit 110.

In the first embodiment, when the data generation apparatus 1 generates new waveform data items by using a waveform data item generated by the waveform data obtaining unit 104 from a biological data item, each of the time shift operation unit 105 and the amplitude operation unit 106 generates the new waveform data items by changing the waveform data item. However, the configuration is not limited to this case. That is, one of the time shift operation unit 105 and the amplitude operation unit 106 may generate new waveform data items.

In addition, in the first embodiment, when the data generation apparatus 1 generates new waveform data items using a waveform data item generated by the waveform data obtaining unit 104, one of the time shift operation unit 105 and the amplitude operation unit 106 generates the new waveform data items by changing the waveform data item. However, the configuration is not limited to this example. Both the time shift operation unit 105 and the amplitude operation unit 106 may generate a new waveform data item by changing the waveform data item. Such a new waveform data item is a waveform data item that is shifted from the original waveform data item along the time axis and whose amplitude is changed relative to the amplitude of the original waveform data item.

1-3. Advantages, Etc.

Regarding the data generation apparatus 1 according to the first embodiment described above, a fluctuation of the latency of an event-related potential relates to an internal factor of the user, and a fluctuation of the amplitude of an event-related potential relates to an external factor of the user. New waveform data items generated by the data generation apparatus 1 using time change values from a waveform data item of a biological data item simulate fluctuations of the waveform data item of the biological data item that result from the internal factor of the user. New waveform data items generated by the data generation apparatus 1 using amplitude change values from the waveform data item of the biological data item simulate fluctuations of the waveform data item of the biological data item that result from the external factor of the user. Since these new waveform data items have a label identical to the label of the waveform data item of the biological data item, these new waveform data items can be pseudo data items of the waveform data item of the biological data item. Thus, the data generation apparatus 1 enables generation of pseudo biological data items from an existing biological data item and consequently enables generation of a large amount of biological training data items.

In addition, the new waveform data items generated by the data generation apparatus 1 according to the first embodiment from the waveform data item of the biological data item can be pseudo data items of the event interval of the waveform data item of the biological data item. Thus, the new waveform data items can virtually represent the states in which an event occurs.

In addition, with the data generation apparatus 1 according to the first embodiment, an amount of training data that is necessary for generating a classifier having a sufficient performance can be obtained. This can consequently reduce the number of trials and reduce the load imposed on a subject in the case where a biological data obtaining experiment is performed on a person to collect the training data. In particular, when an uncomfortable state such as pain or sorrow is measured, duration of the experiment, that is, the number of trials of the experiment, may be reduced. Thus, data generation using the data generation apparatus 1 is effective.

### Second Embodiment

A data generation apparatus according to a second embodiment will be described. The data generation apparatus according to the second embodiment constitutes a classification system 200 together with the classification apparatus 2. The configuration of the data generation apparatus according to the second embodiment is the same or substantially the same as that of the data generation apparatus 1 according to the first embodiment. Hereinafter, differences from the first embodiment will be mainly described.

2-1. Configuration of Classification System

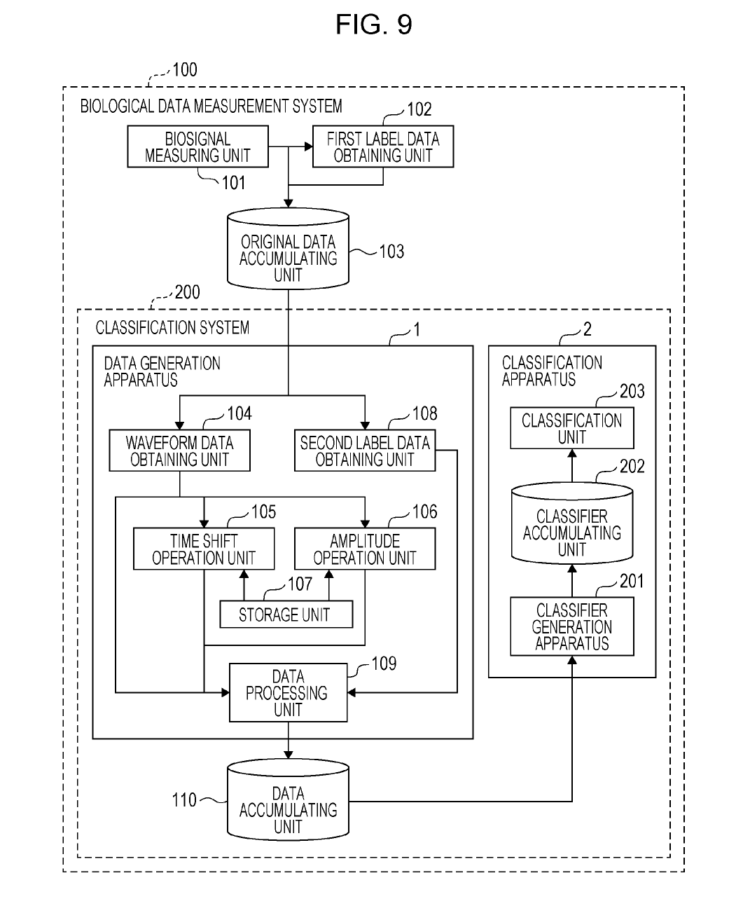

A configuration of the classification system 200 including the data generation apparatus 1 according to the second embodiment will be described. FIG. 9 illustrates a functional configuration of the classification system 200 including the data generation apparatus 1 according to the second embodiment. As illustrated in FIG. 9, the classification system 200 includes the data generation apparatus 1, the classification apparatus 2, and the data accumulating unit 110. The classification apparatus 2 includes a classifier generation apparatus 201, a classifier accumulating unit 202, and a classification unit 203.

The classifier generation apparatus 201 generates a classifier by using waveform data items stored in the data accumulating unit 110 as training data. The classifier generation apparatus 201 receives waveform data items each associated with a label as input data. Then, the classifier generation apparatus 201 trains a classifier, that is, reconstructs the classifier, such that a result output by the classifier that has received a waveform data item indicates the label corresponding to the waveform data item. Training the classifier means reconstructing the classifier such that a correct result is output for input data. The classifier generation apparatus 201 uses various waveform data items corresponding to various labels as input data and repeatedly reconstructs the classifier such that the correct label is output, thereby increasing the accuracy of the output of the classifier. The classifier generation apparatus 201 stores, in the classifier accumulating unit 202, the classifier trained by repeated reconstruction. Once the waveform data items stored in the data accumulating unit 110 are updated because of addition of new data items or the like, the classifier generation apparatus 201 may train the classifier by using the waveform data items after the update.

The classifier generation apparatus 201 uses all the waveform data items stored in the data accumulating unit 110 when generating the classifier. The waveform data items used by the classifier generation apparatus 201 include waveform data items generated by the waveform data obtaining unit 104 from a biological data item, waveform data items generated by the time shift operation unit 105 by changing the waveform data items, and waveform data items generated by the amplitude operation unit 106 by changing the waveform data items. That is, the waveform data items used by the classifier generation apparatus 201 include waveform data items obtained by data augmentation.

Although the classifier generation apparatus 201 generates a classifier that uses waveform data items as input data, the classifier generation apparatus 201 may generate a classifier that uses biological data items such as biosignals as input data. Such a classifier may output, in response to input of a biological data item, a label corresponding to the biological data item.

In the second embodiment, a machine learning model is applied to the classifier. Further, the machine learning model applied to the classifier is a neural-network-based machine learning model such as deep learning. However, another learning model may be used. For example, the machine learning model may be a machine learning model using random forest, genetic programming, or the like.

A neural network is an information processing model based on the cranial nervous system. A neural network includes multiple node layers including an input layer and an output layer. Each of the node layers includes one or more nodes. Model information of a neural network represents the number of node layers constituting the neural network, the number of nodes included in each of the node layers, and a type of the entire neural network or of each node layer. A neural network is constituted by, for example, an input layer, one or more intermediate layers, and an output layer. The neural network sequentially performs, for information input to the node of the input layer, processing of outputting the information from the input layer to the intermediate layer, processing of the information in the intermediate layers, processing of outputting the information from the intermediate layer to the output layer, and processing of the information in the output layer, and outputs an output result appropriate for the input information. Note that each node of one layer is connected to each node of the next layer, and connections between nodes are weighted. Information processed by a node of one layer is weighted according to the connection between the nodes and is output to the nodes of the next layer. In neural-network-based learning, weights between nodes are adjusted so that, when a waveform data item is input to the input layer, an appropriate label is output by the output layer.

The classifier accumulating unit 202 is capable of storing information and enables retrieval of the stored information. The classifier accumulating unit 202 is implemented by a storage device mentioned in the description of the original data accumulating unit 103. The classifier accumulating unit 202 stores the classifier output by the classifier generation apparatus 201. The classifier stored in the classifier accumulating unit 202 is used by the classification unit 203 (described later).

The classification unit 203 inputs a biosignal of a user to the classifier stored in the classifier accumulating unit 202 and determines a label corresponding to the biosignal of the user. In the second embodiment, the classification unit 203 inputs a brainwave data item of a user to the classifier and determines a label corresponding to the brainwave data item of the user. The classification unit 203 may obtain a biosignal of a user from the biosignal measuring unit 101 of the biological data measurement system 100 or from a biosignal measurement apparatus provided separately from the biological data measurement system 100.

Components of the classification apparatus 2 including the classifier generation apparatus 201 and the classification unit 203 may be constituted by a computer system (not illustrated) including a processor such as a CPU, a RAM, a ROM, etc. Functions of some or all of the components may be achieved as a result of the CPU executing a program stored in the ROM by using the RAM as a work memory. In addition, functions of some or all of the components mentioned above may be achieved by a dedicated hardware circuit, such as an electronic circuit or an integrated circuit. The program may be stored in the ROM in advance or may be provided as an application via communication through a communication network such as the Internet, via communication based on a mobile communication standard, via a wireless network of another kind, via a wired network, via broadcasting, etc.

In addition, the data generation apparatus 1, the classification apparatus 2, and the data accumulating unit 110 may constitute a single apparatus together or may constitute multiple individual apparatuses. The data generation apparatus 1, the classification apparatus 2, and the data accumulating unit 110 which constitute multiple apparatuses may be connected to each other via wired or wireless communication. The wireless communication may be wireless communication via a communication network such as the Internet. An example of such wireless communication is Wi-Fi (registered trademark). In addition, the classification system 200 may include at least one of the biosignal measuring unit 101, the first label data obtaining unit 102, and the original data accumulating unit 103.

### 2-2. Operation of Classification Apparatus

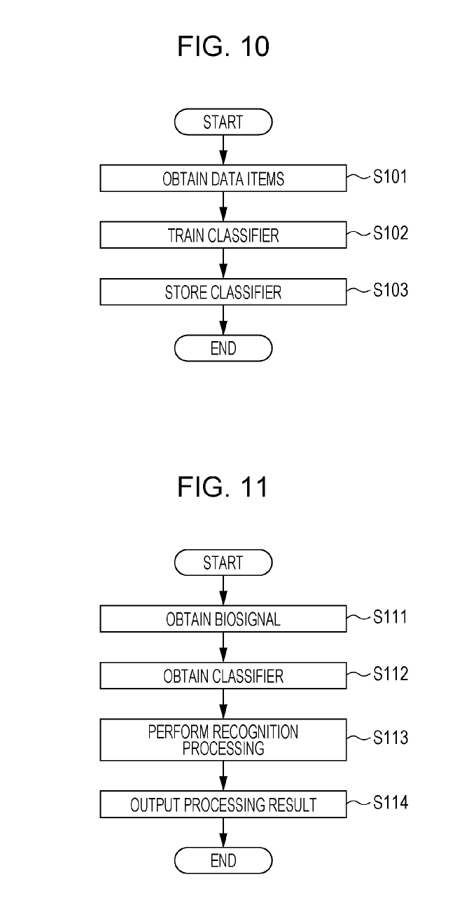

A classifier generation operation performed by the classification apparatus 2 according to the second embodiment will be described next with reference to FIG. 10. FIG. 10 is a flowchart illustrating an example of a flow of the classifier generation operation performed by the classification apparatus 2 according to the second embodiment.

In step S101, the classifier generation apparatus 201 obtains data items stored in the data accumulating unit 110. The data items that are obtained include data items obtained by the data generation apparatus 1 through data augmentation processing. The data items that are obtained each include a waveform data item and label data corresponding to the waveform data item.

In step S102, the classifier generation apparatus 201 trains the classifier stored in the classifier accumulating unit 202 by using the data items obtained from the data accumulating unit 110 as training data.

In step S103, the classifier generation apparatus 201 stores, in the classifier accumulating unit 202, the classifier that is generated (i.e., reconstructed) by training in step S102.

Further, a classification operation performed by the classification apparatus 2 will be described with reference to FIG. 11. FIG. 11 is a flowchart illustrating an example of a flow of the classification operation performed by the classification apparatus 2 according to the second embodiment.

In step S111, the classification unit 203 obtains a biosignal data item of a brainwave, for example. The biosignal data item includes measured values of a biosignal measured from a user using a measurement device (not illustrated). The measured values of the biosignal are measured values obtained at multiple time points and are associated with the respective time points.

In step S112, the classification unit 203 obtains the classifier stored in the classifier accumulating unit 202. Further, in step S113, the classification unit 203 classifies the biosignal data item obtained in step S111 by using the obtained classifier. That is, the classification unit 203 performs biosignal data recognition processing. Specifically, the classification unit 203 inputs the biosignal data item to the classifier and obtains label information output by the classifier.

In step S114, the classification unit 203 outputs the label information obtained in step S113, that is, the recognition result. A target to which the label information is output may be an apparatus connected to the classification apparatus 2. Examples of such an apparatus include a display and/or a speaker. The display may include a liquid crystal panel or an organic or inorganic electroluminescence (EL) display panel.

### 2-3. Application Examples of Classification System

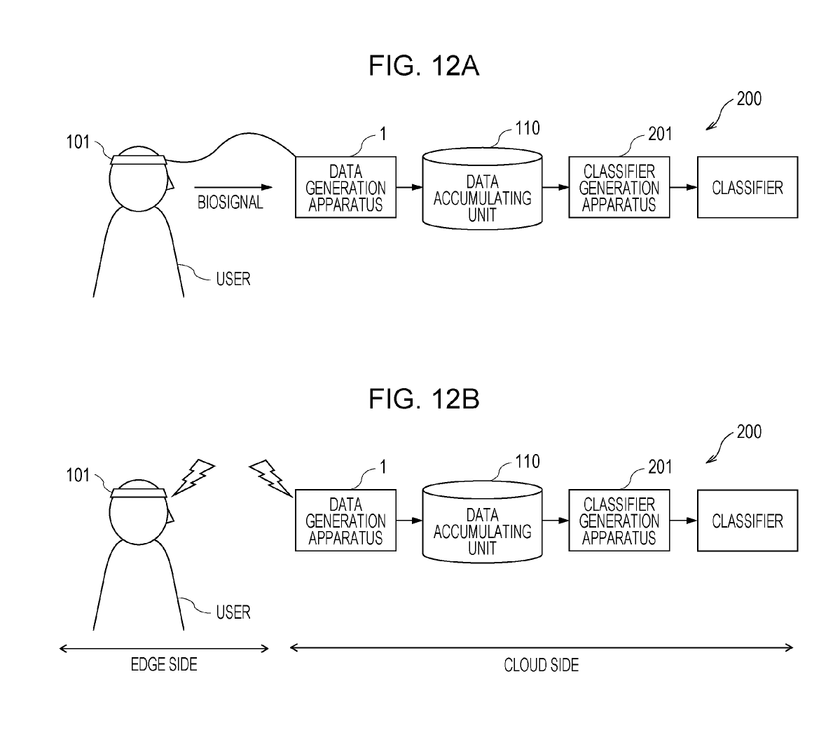

Application examples of the classification system 200 will be described next. FIG. 12A illustrates one of the application examples of the classification system 200. In the example illustrated in FIG. 12A, the biosignal measuring unit 101, the data generation apparatus 1, the data accumulating unit 110, and the classifier generation apparatus 201 are connected to each other with a cable. For example, when an application for determining a psychological state of a user from the activity of the brain is created, the application may be customized for each user because the activity of the brain differs from user to user. Thus, the biosignal measuring unit 101 is attached to various users and measures biological data items when the various users perform a task. The data generation apparatus 1 performs data augmentation on the measured biological data items and stores resultant data items in the data accumulating unit 110. The classifier generation apparatus 201 generates a classifier by training the classifier using the data items stored in the data accumulating unit 110.

FIG. 12B illustrates another application example of the classification system 200. As illustrated in FIG. 12B, the data generation apparatus 1, the data accumulating unit 110, and the classifier generation apparatus 201 may constitute a cloud system. In this case, the biosignal measuring unit 101 that is located on the edge side and the data generation apparatus 1, the data accumulating unit 110, and the classifier generation apparatus 201 that are located on the cloud side may mutually exchange information via a communication network such as the Internet.

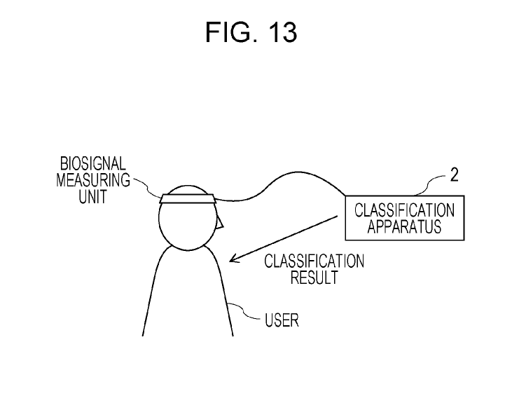

FIG. 13 illustrates one of application examples of the classification apparatus 2. As illustrated in FIG. 13, the classification apparatus 2 is connected, with a cable, to the biosignal measuring unit attached to the user. The biosignal measuring unit may be included in or separated from the biological data measurement system 100. The classification apparatus 2 obtains a biosignal from a user and determines a label of the biosignal. For example, when an application for detecting pain of a user from a brainwave is created, the classification apparatus 2 obtains a measured brainwave data item using the biosignal measuring unit and outputs a determined result of the psychological state regarding pain. Examples of the psychological state regarding pain include psychological states of “pain” and “pain-free”.

### 2-4. Advantages, Etc.

The classification system 200 according to the second embodiment described above is capable of generating pseudo waveform data items corresponding to a label from a waveform data item of an existing biological data item. The classification system 200 is also capable of generating a classifier using many biological data items including existing biological data items and pseudo biological data items. Thus, the classification system 200 enables the classifier to have an improved performance. In addition, the classifier generated using biological data items associated with various labels is capable of classifying various labels from biological data items.

In addition, the classification system 200 according to the second embodiment is also applicable to biometric authentication that requires an association between biometric information and a person and can implement an easy-to-perform calibration in such biometric authentication.

### Third Embodiment

A data generation apparatus 21 according to a third embodiment will be described. The data generation apparatus 21 according to the third embodiment includes a peak detection unit 121 and a peak-emphasis operation unit 122 in place of the amplitude operation unit 106 included in the data generation apparatus 1 according to the first embodiment. Differences from the first and second embodiments will be mainly described below.

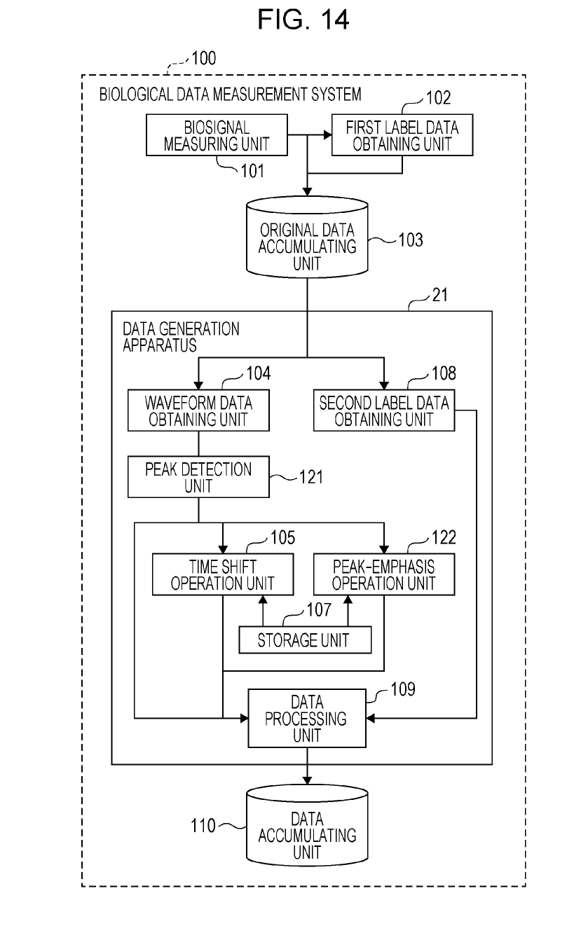

FIG. 14 illustrates a functional configuration of the data generation apparatus 21 according to the third embodiment. As illustrated in FIG. 14, the data generation apparatus 21 according to the third embodiment includes the waveform data obtaining unit 104, the peak detection unit 121, the time shift operation unit 105, the peak-emphasis operation unit 122, the storage unit 107, the second label data obtaining unit 108, and the data processing unit 109. The data generation apparatus 1 according to the first embodiment generates new waveform data items by changing the entire waveform of a waveform data item of an event interval. In contrast, the data generation apparatus 21 according to the third embodiment determines a peak portion to be focused on in a waveform of a waveform data item of an event interval and changes the waveform data item around the determined peak portion to generate new waveform data items.

The peak detection unit 121 detects the largest value of a bioelectric potential in an event interval and a time point of the largest value in a waveform data item generated by the waveform data obtaining unit 104. The largest value is a local maximum value, and the time point of the largest value corresponds to the latency. For example, when there are multiple largest values, the peak detection unit 121 may select the largest value that appears the earliest.

More specifically, the peak detection unit 121 obtains multiple waveform data items associated with the same task, that is, the same label, and calculates an average waveform data item obtained by averaging the waveform data items. The average waveform data item represents an average of results of multiple trials of a single task and is also referred to as a trial average waveform data item.

In calculation of the trial average waveform data item, an average of bioelectric potentials of the multiple waveform data items at an identical time point from the start point of an event interval is calculated, and the trial average waveform data item is generated using the averages at respective time points. The peak detection unit 121 detects the largest value of the bioelectric potential in the event interval and the time point of the largest value in the trial average waveform data item. At that time, the peak detection unit 121 detects the largest value in an interval that starts after a predetermined period elapses from a timing at which a stimulus occurs, that is, the start point of the event interval. The predetermined period can be determined in accordance with a target bioelectric potential. In the case where the target bioelectric potential is the brainwave, an example of the predetermined period is 200 ms. The predetermined period may be set to be equal in the case where the stimulus received by the user is an external stimulus and in the case where the stimulus received by the user is an internal stimulus. Further, the peak detection unit 121 sets, as a waveform data change interval, an interval of 200 ms before and after the time point of the largest value which is the peak, i.e., an interval of 400 ms. Note that the duration of the waveform data change interval is not limited to 400 ms and may be another value.

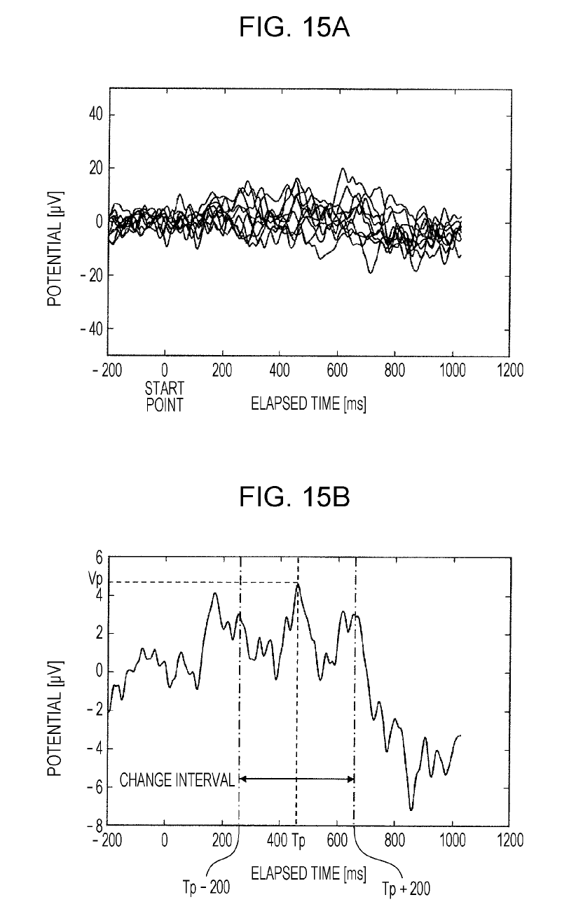

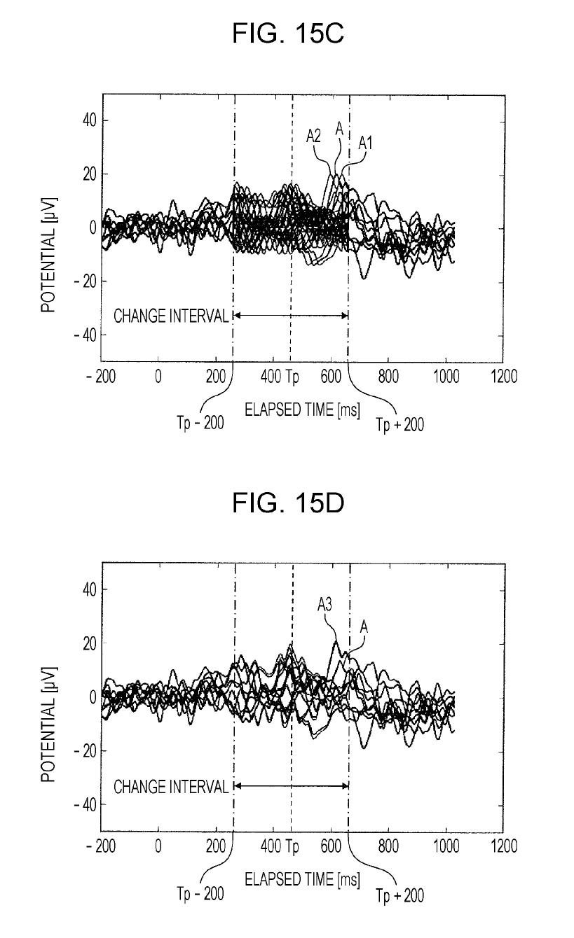

For example, FIG. 15A illustrates an example of multiple waveform data items of brainwaves generated by the waveform data obtaining unit 104. FIG. 15A illustrates waveform data items for ten trials of a task. The peak detection unit 121 averages the waveform data items for ten trials of the task illustrated in FIG. 15A to obtain a trial average waveform data item illustrated in FIG. 15B. The peak detection unit 121 further identifies the largest value Vp (unit: μV) of the bioelectric potential in an interval after the elapsed time of 200 ms in the trial average waveform data item. The peak detection unit 121 then identifies the elapsed time Tp (unit: ms) at which the bioelectric potential shows the largest value Vp. The peak detection unit 121 sets an interval from (Tp−200) ms to (Tp+200) ms as the waveform data change interval.

The time shift operation unit 105 generates new waveform data items by shifting each waveform data item used by the peak detection unit 121 in calculation of the trial average waveform data item by a time change value set in advance in a positive direction and/or a negative direction of the time axis. At that time, the time shift operation unit 105 shifts a portion of each waveform data item in the change interval calculated by the peak detection unit 121 by the time change value. Processing that is performed for a portion of the waveform data item that is moved to be outside the change interval around the start and the end of the change interval is substantially the same as that of the first embodiment.

For example, FIG. 15C illustrates an example of new waveform data items generated by shifting the waveform data items illustrated in FIG. 15A in the change interval along the time axis. In the example illustrated in FIG. 15C, the time shift operation unit 105 shifts the waveform data items illustrated in FIG. 15A in the change interval by ±20 ms. For example, by shifting the waveform data item A in the change interval, a waveform data item A1 corresponding to shifting by +20 ms and a waveform data item A2 corresponding to shifting by −20 ms are generated.

The peak-emphasis operation unit 122 generates new data items by changing the amplitude of each waveform data item, used by the peak detection unit 121 in calculation of the trial average waveform data item, in the change interval calculated by the peak detection unit 121, that is, by multiplying the amplitude by an amplitude change value set in advance. The amplitude changing method used by the peak-emphasis operation unit 122 is substantially the same as that used by the amplitude operation unit 106 according to the first embodiment.

For example, the peak-emphasis operation unit 122 performs the processing in accordance with a table such as Table 3 below. Table 3 shows multiplying factors of the quantity of waveform data items after data augmentation relative to the quantity of waveform data items before data augmentation. Further, for each of the multiplying factors, amplitude change values are set. In the third embodiment, the amplitude change value is set to, but not limited to, a value exceeding 1 in order to emphasize the peak portion of each waveform data item. Note that Table 3 shows merely an example of the generation rule, and the generation rule is not limited to this example.

TABLE 3
Relationship between Amplitude Change Values and Data Increasing Rate

| | | Amplitude Change Values (Unit: times) | | | | | |
|---|---|---|---|---|---|---|---|
| Data | ×1 | 1 | | | | | |
| Increasing | ×3 | 1 | 1 + n/100 | 1 + 2n/100 | | | |
| Rate | ×5 | 1 | 1 + n/100 | 1 + 2n/100 | 1 + 3n/100 | 1 + 4n/100 | |
| | ×7 | 1 | 1 + n/100 | 1 + 2n/100 | 1 + 3n/100 | 1 + 4n/100 | 1 + 5n/100 |
| | | | 1 + 6n/100 | | | | |
| | ×9 | 1 | 1 + n/100 | 1 + 2n/100 | 1 + 3n/100 | 1 + 4n/100 | 1 + 5n/100 |
| | | | 1 + 6n/100 | 1 + 7n/100 | 1 + 8n/100 | | |
| | ×11 | 1 | 1 + n/100 | 1 + 2n/100 | 1 + 3n/100 | 1 + 4n/100 | 1 + 5n/100 |
| | | | 1 + 6n/100 | 1 + 7n/100 | 1 + 8n/100 | 1 + 9n/100 | 1 + 10n/100 |

FIG. 15D illustrates an example of new waveform data items generated by changing the amplitudes of the waveform data items illustrated in FIG. 15A in the change interval. In the example illustrated in FIG. 15D, the peak-emphasis operation unit 122 multiplies the amplitudes of the waveform data items illustrated in FIG. 15A in the change interval by 1.2. For example, as a result of changing the amplitude of the waveform data item A in the change interval, a waveform data item A3 is generated. In the example illustrated in FIG. 15D, the multiplying factor of the amplitude of the waveform data item A in the change interval is not constantly set to 1.2 but changes in order to suppress the waveform data item A3 from becoming discontinuous at the start point and the end point of the change interval. The multiplying factor is set to 1 around the start point and the end point of the change interval, is set to 1.2 around a middle position of the change interval, and changes at a predetermined change rate from 1 to 1.2 between the start point or the end point and the middle position. The predetermined change rate may be constant between the start point or the end point and the middle position or may change smoothly to form a curve defined by a function. In addition, the middle position may be a midpoint of the start point and the end point, that is, the position of the peak of the waveform of the trial average waveform data item, or may be another position. Note that the multiplying factor of the amplitude of the waveform data item in the change interval may be constant. For example, in the cases where the multiplying factor is small and the amplitude is small at the start point and the end point of the change interval, continuity of a waveform data item obtained by changing the amplitude can be maintained even if the multiplying factor is constant. The new waveform data items generated by the peak-emphasis operation unit 122 in the above manner are waveform data items whose peak portion is emphasized.

Although the peak detection unit 121 calculates the trial average waveform data item using all the obtained waveform data items associated with the same label, the configuration is not limited to this one. The trial average waveform data item may be calculated from one or more waveform data items among the obtained waveform data items.

### EXAMPLES

A description will be given of details and advantages of an experiment in which data augmentation was performed by using the classification system 200 according to the second embodiment and recognition was performed by a classifier that was trained using data items resulting from the data augmentation.

Data items used in the experiment were brainwave data items obtained when five subjects performed two types of tasks. The two types of tasks included a “motivating” task for which the subject can actively take an action and a “non-motivating” task for which the subject cannot actively take an action. The “motivating” task is an assignment in which the subject looks at a clock and presses a button after five seconds from a start timing set in advance. The “non-motivating” task is an assignment in which the subject presses a button upon confirming that a clock has stopped after five seconds from a start timing set in advance. As described above, the two types of tasks were associated with the motivation state of the subject, and each brainwave data item was associated with the corresponding task by labeling with “motivating” or “non-motivating”.

A brainwave measurement experiment was performed on five subjects who performed the tasks in order to obtain brainwave data items. In the brainwave measurement experiment, each subject performed each of the two types of tasks 30 times (trials), that is, performed 60 trials of the tasks in total. Each of the measured brainwave data items was sectioned for each trial and was further subjected to noise removal using a bandpass filter and baseline correction (also called 0-correction). To remove outliers of the bioelectric potential, a reference value (60 μV) was set as the threshold, and trials in which the bioelectric potential of the reference value or greater was detected were removed. As a result, the average number of task trials of the five subjects was equal to 58.6.

Then, a classifier that distinguishes between the two types of tasks, i.e., the “motivating” and “non-motivating” tasks from a brainwave data item was generated by the classification system 200 for each subject by using such brainwave data items of the corresponding subject in learning. Each classifier was generated through deep learning using a neural network constituted by an input layer, two intermediate layers, and an output layer. Further, as classifiers for each subject, a classifier according to the example and classifiers according to comparative examples were generated. The classifier according to the example was generated using data items obtained by data augmentation performed on the brainwave data items by the data generation apparatus 1. The classifiers according to the comparative examples were generated using original brainwave data items not resulting from data augmentation. The classifiers according to the comparative examples included a classifier according to a first comparative example generated using a sufficient amount of brainwave data items, a classifier according to a second comparative example generated using an insufficient amount of brainwave data items, and a classifier according to a third comparative example generated using an intermediate amount of brainwave data items between the amount of data used in the first comparative example and the amount of data used in the second comparative example.

The sufficient amount of brainwave data items indicates all the brainwave data items obtained through the target trials. In this experiment, the brainwave data items of the target trials were brainwave data items obtained for an average of 58.6 trials. Specifically, the brainwave data items of the target trials were all the brainwave data items of the remaining number of trials after removing the brainwave data items of the trials including bioelectric potential outliers from the brainwave data items of 60 trials. Hereinafter, such brainwave data items are referred to as brainwave data items of all trials. The insufficient amount of brainwave data items indicates brainwave data items of 20 trials extracted at random from among the brainwave data items of the target trials. The intermediate amount of data items between the amounts used in the first and second comparative examples indicates brainwave data items of 40 trials extracted at random from among the brainwave data items of the target trials. In the brainwave data items of 20 trials, the number of trials for the brainwave data items associated with the “motivating” label was set equal to the number of trials for the brainwave data items associated with the “non-motivating” label. The same applies to the brainwave data items of 40 trials.

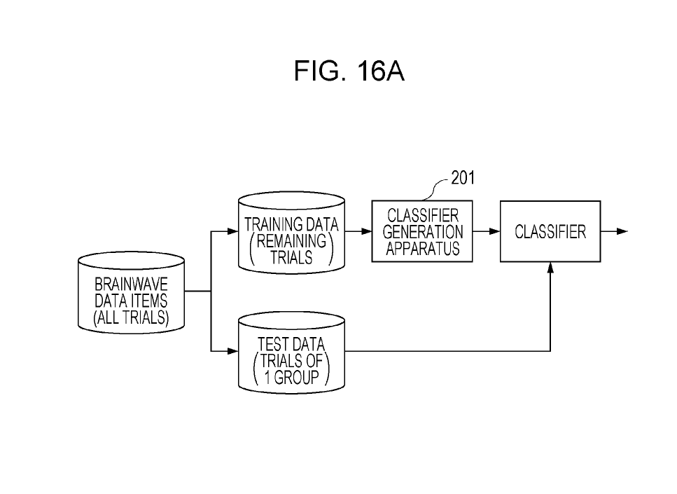

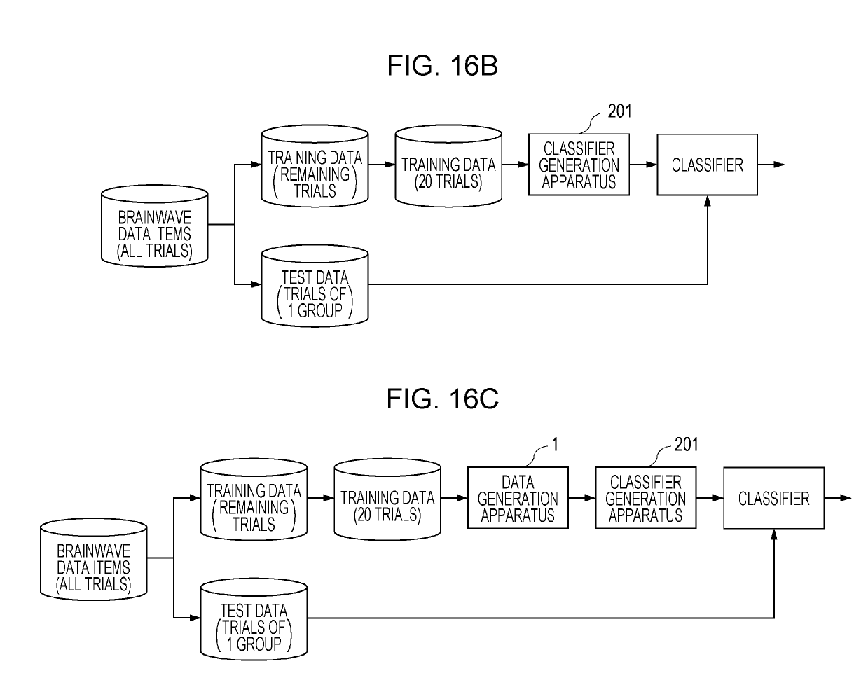

FIGS. 16A to 16C illustrate data assignment methods used when the classifiers according to the first comparative example, the second comparative example, and the example were generated. In this experiment, 10-fold cross-validation was used as an evaluation method of machine learning of the classifier. According to this evaluation method, brainwave data items are divided into ten groups, and one of the ten groups is used as test data and the remaining nine groups are used as training data. Each classifier was generated using the brainwave data items of the remaining nine groups. Generation of the classifier was repeatedly performed ten times while switching between the groups serving as the test data so that every group played the role of the test data. Then, the classification performance of the classifier for the test data was evaluated. Specifically, the performance of the classifier was evaluated from the classification performance for the test data of the ten groups. In this experiment, a classification rate was used as an index representing the classification performance. Thus, the classification rate of each classifier was calculated for the test data of all the ten groups regarding the brainwave data items of each subject. Then, the classification rates of the classifiers for the respective subjects were averaged.

FIG. 16A illustrates an example of the case of the classifier according to the first comparative example. The classifier according to the first comparative example uses, as training data, all the brainwave data items of nine groups among the brainwave data items of all trials. FIG. 16B illustrates an example of the case of the classifier according to the second comparative example. The classifier according to the second comparative example uses, as training data, brainwave data items of 20 trials among the brainwave data items of nine groups out of the brainwave data items of all trials. In the case of the third comparative example, the classifier uses, as training data, brainwave data items of 40 trials among the brainwave data items of nine groups out of the brainwave data items of all trials.

FIG. 16C illustrates an example of the case of the classifier according to the example. In this case, brainwave data items of 20 trials are extracted from among the brainwave data items of nine groups out of the brainwave data items of all trials. The data generation apparatus 1 performs data augmentation on the extracted brainwave data items of 20 trials to increase the amount of data. The classifier according to the example uses the resulting data items as training data. In this experiment, data was assigned so that the same test data was used for four classifiers, i.e., the classifiers according to the example and the first to third comparative examples for each of the subjects.

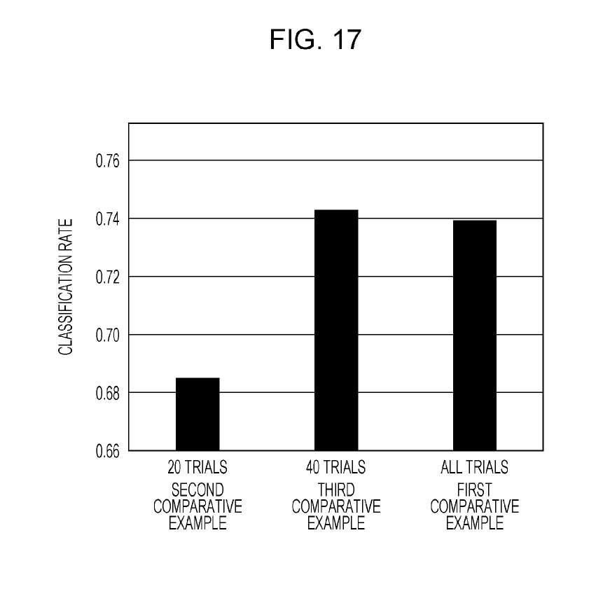

The classification performance of each of the classifiers according to the example and the first to third comparative examples was determined as described below. FIG. 17 illustrates the classification rates of the classifiers according to the first to third comparative examples. Specifically, an average classification rate of the classifiers for the five subjects according to each of the first to third comparative examples is illustrated. The classification rates of the classifiers according to the first and third comparative examples were equal to 73.9% and 74.3%, respectively. The classification rate of the classifier according to the second comparative example was equal to 68.5%, which is greatly lower than those of the classifiers according to the first and third comparative examples. Accordingly, it is admitted that the classification rate of the classifier decreases as a result of decreasing the number of training data items.

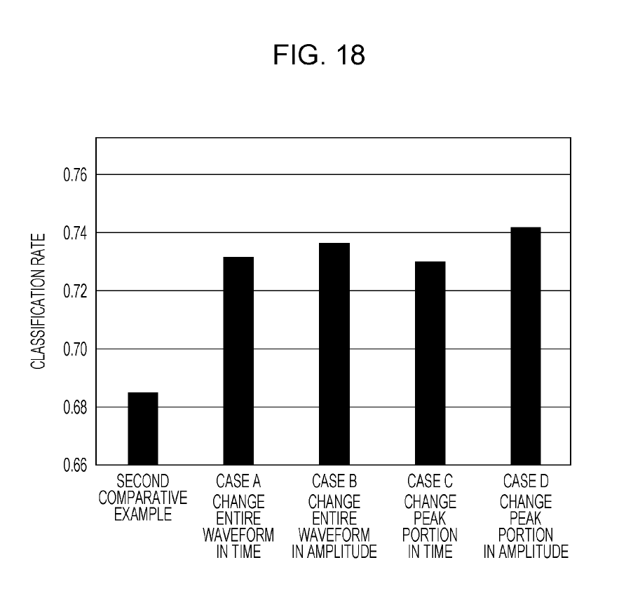

FIG. 18 illustrates the classification rates of the classifiers according to the example and the second comparative example. As in FIG. 17, an average classification rate of the classifiers for the five subjects according to each of the example and the second comparative example is illustrated. Specifically, as for the classifier according to the example, FIG. 18 illustrates classification rates in cases A to D in which the classifier is generated using four different kinds of training data.

The training data used in the case A includes brainwave data items generated by the data generation apparatus 1 by shifting the entire waveform of the waveform data items of the brainwave data items of 20 trials along the time axis. That is, the training data used in the case of A includes data items obtained by performing data augmentation on the entire waveforms of waveform data items of brainwave data items in terms of time.

The training data used in the case B includes brainwave data items generated by the data generation apparatus 1 by changing the amplitudes of the entire waveforms of the waveform data items of the brainwave data items of 20 trials. That is, the training data used in the case B includes data items obtained by performing data augmentation on the entire waveforms of waveform data items of brainwave data items in terms of amplitude.

The training data used in the case C includes brainwave data items generated by the data generation apparatus 1 by shifting the peak portions, i.e., the change intervals, of the waveforms of the waveform data items of the brainwave data items of 20 trials along the time axis. That is, the training data used in the case C includes data items obtained by performing data augmentation on the peak portions of waveforms of waveform data items of brainwave data items in terms of time.

The training data used in the case D includes brainwave data items generated by the data generation apparatus 1 by changing the amplitudes of the peak portions of the waveforms of the waveform data items of the brainwave data items of 20 trials. That is, the training data used in the case D includes data items obtained by performing data augmentation on the peak portions of waveforms of wave data items of brainwave data items in terms of amplitude.

In the cases A and C, Table 4 which is the same as Table 1 above is used to set the time change values. Specifically, in the cases where the value of “n” is set to values of 5-ms intervals from 5 ms to 50 ms, that is, to 5, 10, 15, 20, 25, 30, 35, 40, 45, and 50, Table 4 of each of the cases is generated. That is, ten kinds of Table 4 are generated, and the time change values included in the ten kinds of Table 4 are used in data augmentation. Specifically, for each of the ten kinds of Table 4, the quantity of brainwave data items is increased by five increasing rates of ×3, ×5, ×7, ×9, and ×11 using the time change values set for the respective increasing rates. Consequently, for each of the ten kinds of Table 4, five sets of data items are generated by data augmentation. In each of the cases A and C, 50 (the number of kinds of Table 4: 10 kinds x the number of kinds of increasing rates: 5 kinds) classifiers were generated by using, as the training data, data items increased to respective increasing rates. FIG. 18 illustrates the average classification rate of the 50 classifiers in each of the cases A and C.

TABLE 4
Relationship between Time Change
Values and Data Increasing Rate

| | | Time Change Values (Unit: ms) | | | | | |
|---|---|---|---|---|---|---|---|
| Data | ×1 | 0 | | | | | |
| Increasing | ×3 | 0 | ±n | | | | |
| Rate | ×5 | 0 | ±n | ±2n | | | |
| | ×7 | 0 | ±n | ±2n | ±3n | | |
| | ×9 | 0 | ±n | ±2n | ±3n | ±4n | |
| | ×11 | 0 | ±n | ±2n | ±3n | ±4n | ±5n |

In the case B, Table 5 which is the same as Table 2 above is used to set the amplitude change values. Specifically, in the cases where the value of “n” in Table 5 is set to values of 5, 10, 15, 20, 25, 30, 35, 40, 45, and 50 as in the cases A and C, Table 5 is generated for each of the cases. That is, ten kinds of Table 5 are generated, and the amplitude change values included in the ten kinds of Table 5 are used in data augmentation. At that time, amplitude change values that are in a range from 0.1 times to 1.9 times are used among the resulting amplitude change values. Thus, ten amplitude change values for the data increasing rate of ×3, nine amplitude change values for the data increasing rate of ×5, six amplitude change values for the data increasing rate of ×7, four amplitude change values for the data increasing rate of ×9, and three amplitude change values for the data increasing rate of ×11 were used in the ten kinds of Table 5. Consequently, 32 classifiers were generated. FIG. 18 illustrates an average classification rate of the 32 classifiers in the case B.

TABLE 5
Relationship between Amplitude Change Values and Data Increasing Rate

| | | Amplitude Change Values (Unit: times) | | | | | |
|---|---|---|---|---|---|---|---|
| Data | ×1 | 1 | | | | | |
| Increasing | ×3 | 1 | 1 ± n/100 | | | | |
| Rate | ×5 | 1 | 1 ± n/100 | 1 ± 2n/100 | | | |
| | ×7 | 1 | 1 ± n/100 | 1 ± 2n/100 | 1 ± 3n/100 | | |
| | ×9 | 1 | 1 ± n/100 | 1 ± 2n/100 | 1 ± 3n/100 | 1 ± 4n/100 | |
| | ×11 | 1 | 1 ± n/100 | 1 ± 2n/100 | 1 ± 3n/100 | 1 ± 4n/100 | 1 ± 5n/100 |

In the case D, Table 6 which is the same as Table 3 above is used to set the amplitude change values. Specifically, in the cases where the value of “n” in Table 6 is set to values of 5, 10, 15, 20, 25, 30, 35, 40, 45, and 50 as in the cases A and C, Table 6 is generated for each of the cases. That is, ten kinds of Table 6 are generated, and the amplitude change values included in the ten kinds of Table 6 are used in data augmentation. By using the data items increased to each of the increasing rates as the training data, 50 (the number of kinds of Table 6: 10 kinds × the number of kinds of increasing rates: 5 kinds) classifiers were generated. FIG. 18 illustrates an average classification rate of the 50 classifiers in the case D.

TABLE 6
Relationship between Amplitude Change Values and Data Increasing Rate

| | | Amplitude Change Values (Unit: times) | | | | | |
|---|---|---|---|---|---|---|---|
| Data | ×1 | 1 | | | | | |
| Increasing | ×3 | 1 | 1 + n/100 | 1 + 2n/100 | | | |
| Rate | ×5 | 1 | 1 + n/100 | 1 + 2n/100 | 1 + 3n/100 | 1 + 4n/100 | |
| | ×7 | 1 | 1 + n/100 | 1 + 2n/100 | 1 + 3n/100 | 1 + 4n/100 | 1 + 5n/100 |
| | | | 1 + 6n/100 | | | | |
| | ×9 | 1 | 1 + n/100 | 1 + 2n/100 | 1 + 3n/100 | 1 + 4n/100 | 1 + 5n/100 |
| | | | 1 + 6n/100 | 1 + 7n/100 | 1 + 8n/100 | | |
| | ×11 | 1 | 1 + n/100 | 1 + 2n/100 | 1 + 3n/100 | 1 + 4n/100 | 1 + 5n/100 |
| | | | 1 + 6n/100 | 1 + 7n/100 | 1 + 8n/100 | 1 + 9n/100 | 1 + 10n/100 |

As illustrated in FIG. 18, the classification rate of the classifier according to the second comparative example is equal to 68.5%. In contrast, the classification rates of the classifiers according to the example in the cases A to D are increased to be in a range from 73.0% to 74.2%.

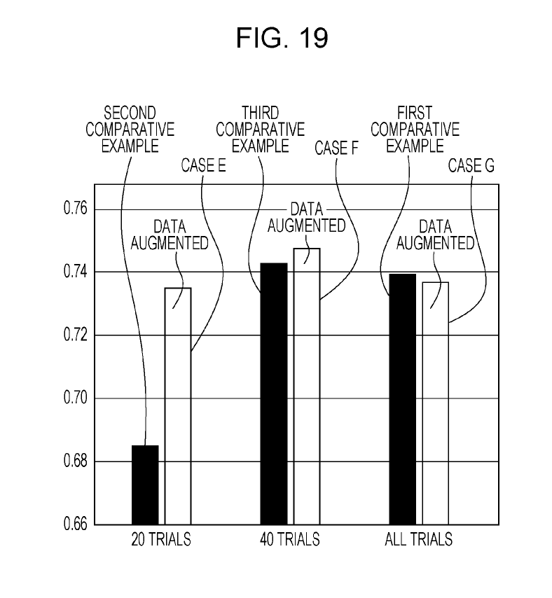

FIG. 19 illustrates the classification rates of the classifiers according to the example and the first to third comparative examples. As in FIG. 17, FIG. 19 illustrates an average classification rate of the classifiers for the five subjects according to each of the example and the first to third comparative examples. Specifically, FIG. 19 illustrates classification rates in cases E to G in which the classifiers according to the example are generated using three different kinds of training data.

The training data used in the case E is data items obtained by the data generation apparatus 1 by performing data augmentation on brainwave data items of 20 trials. The training data used in the case F is data items obtained by the data generation apparatus 1 by performing data augmentation on brainwave data items of 40 trials. The training data used in the case G is data items obtained by the data generation apparatus 1 by performing data augmentation on brainwave data items of all trials. In each of the cases E to G, the data generation apparatus 1 performed data augmentation using the four methods described in the respective cases A to D above. Further, in each of the cases E to G, classifiers were generated using the data items obtained by data augmentation using the four methods as in the cases A to D, and the average classification rate of all the classifiers was calculated. That is, in each of the cases E to G, the processing of the cases A to D was performed and the average classification rate of all the classifiers was calculated. The average classification rates described above are illustrated in the respective cases E to G in FIG. 19.

In the case of 20 trials for which the amount of data is insufficient, the performance of the classifier according to the example in the case E greatly improved compared with that of the classifier according to the second comparative example. However, in the case of all trials for which the amount of data is sufficient, the classification rate of the classifier according to the example in the case G is equal to 73.7%, which is lower than 73.9% that is the classification rate of the classifier according to the first comparative example. In addition, in the case of 40 trials which is between 20 trials and all trials, the classification rate of the classifier according to the example in the case F is equal to 74.8%, which is higher than 74.3% that is the classification rate of the classifier according to the third comparative example but the change is 0.64% and is small. The data augmentation methods of the four cases A to D are collectively summarized from the foregoing that data augmentation performed by the data generation apparatus 1 admittedly provides a great effect on improvement of the performance of a classifier in the case where an amount of actually measured data is not sufficient. In addition, in the other cases, data augmentation performed by the data generation apparatus 1 admittedly enables generation of a classifier having the performance substantially the same as that achieved in the case where actually measured data is used.

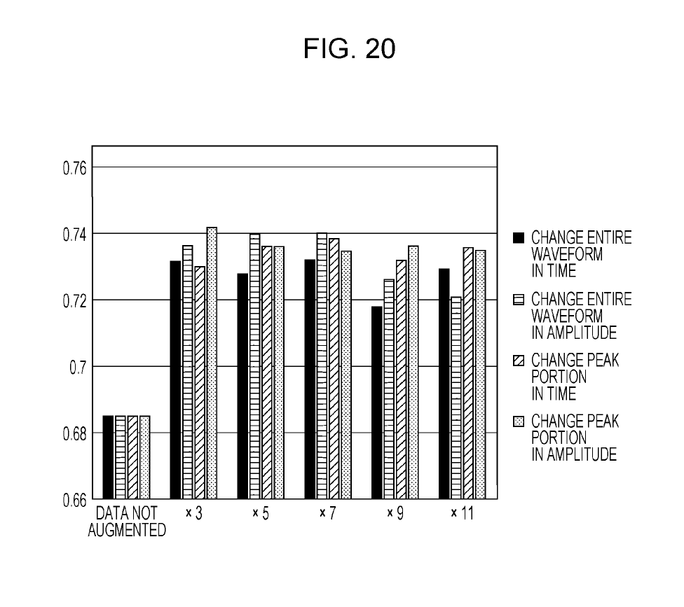

FIG. 20 illustrates a relationship between a data increasing rate achieved by the data generation apparatus 1 and a classification rate of a classifier. FIG. 20 illustrates an example in which the data generation apparatus 1 increases the amount of brainwave data items of 20 trials by three times, five times, seven times, nine times, and eleven times by data augmentation. FIG. 20 also illustrates the classification rates of classifiers generated using data items obtained by data augmentation performed using the above methods of the four cases A to D for each multiplying factor. As in FIGS. 17 to 19, FIG. 20 illustrates the average classification rate of the classifiers for the five subjects.

The classification rate of the classifier generated using brainwave data items of 20 trials is equal to 68.5%. In contrast, the classification rate of each of the classifiers generated using data items obtained by data augmentation exceeds 68.5%. In particular, in the case where data augmentation is performed for the entire waveform in terms of time, which corresponds to the case A, the classification rate is equal to 73.2% when the multiplying factor of the data is ×7. In addition, in the case where data augmentation is performed for the entire waveform in terms of amplitude, which corresponds to the case B, the classification rate is equal to 74.0% when the multiplying factor of the data is ×7. In addition, in the case where data augmentation is performed for a peak portion of the waveform in terms of time, which corresponds to the case C, the classification rate is equal to 73.8% when the multiplying factor of the data is ×7. Further, in the case where data augmentation is performed for a peak portion of the waveform in terms of amplitude, which corresponds to the case D, the classification rate is equal to 74.2% when the multiplying factor of the data is ×3. In this case, the classification performance of the classifier improves the most.

FIG. 20 indicates that the effect of data augmentation for the entire waveform in terms of time and amplitude on the improvement of the performance of the classifier tends to decrease as the multiplying factor of the data increases. It is considered that this occurs because of an influence of the bioelectric potential located at a position separate from the peak, which is less related to the fluctuation in the event-related potential, being changed into data having characteristics that are greatly different from original characteristics of this bioelectric potential.

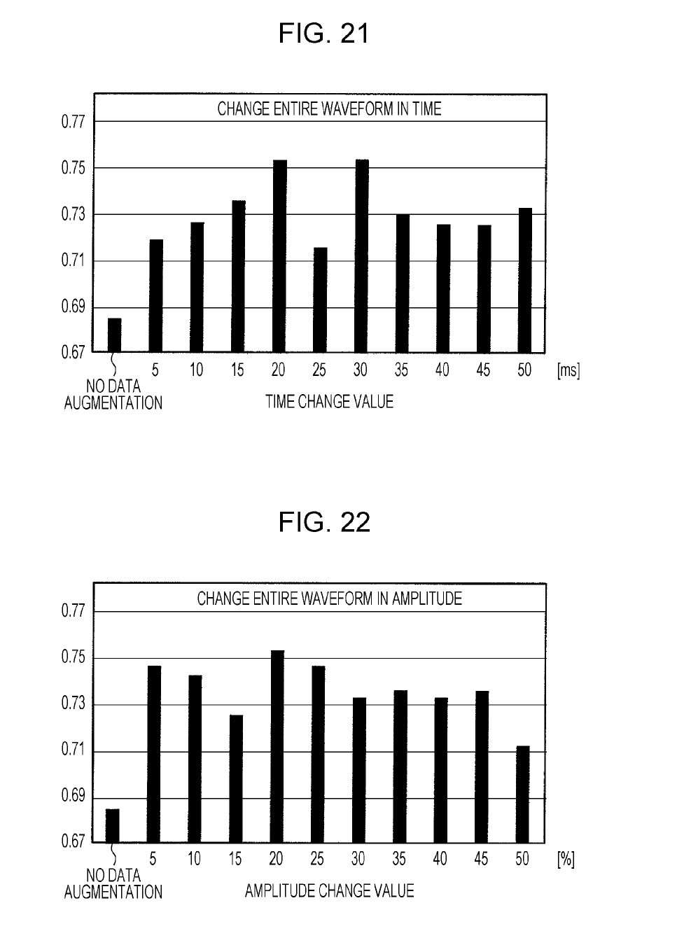

FIGS. 21 to 24 each illustrate a relationship between a time change value or an amplitude change value used for waveform data items and a classification rate of a classifier in the case where the data generation apparatus 1 increases the amount of brainwave data items of 20 trials by three times through data augmentation. As in FIGS. 17 to 19, FIGS. 21 to 24 each illustrate the average classification rate of the classifiers for the five subjects.

FIG. 21 illustrates the case where data augmentation is performed for the entire waveform in terms of time, which corresponds to the case A. FIG. 21 illustrates the classification rates of classifiers generated using data items obtained by data augmentation performed using the time change value of “±n” for the data increasing rate of ×3 in Table 1 above. The classification rates of the classifiers in ten cases where “n” is equal to 5, 10, 15, 20, 25, 30, 35, 40, 45, and 50 are compared with the case where data augmentation is not performed on the brainwave data items of 20 trials. The numerals of the horizontal axis of FIG. 21 indicate the value of “n”.

FIG. 22 illustrates the case where data augmentation is performed for the entire waveform in terms of amplitude, which corresponds to the case B. FIG. 22 illustrates the classification rates of classifiers generated using data items obtained by data augmentation performed using the amplitude change value of “1±n/100” times for the data increasing ratio of ×3 in Table 2 above. The classification rates of the classifiers in ten cases where “n” is equal to 5, 10, 15, 20, 25, 30, 35, 40, 45, and 50 are compared with the case where data augmentation is not performed on the brainwave data items of 20 trials. The numerals of the horizontal axis of FIG. 22 indicate the value of “n”.

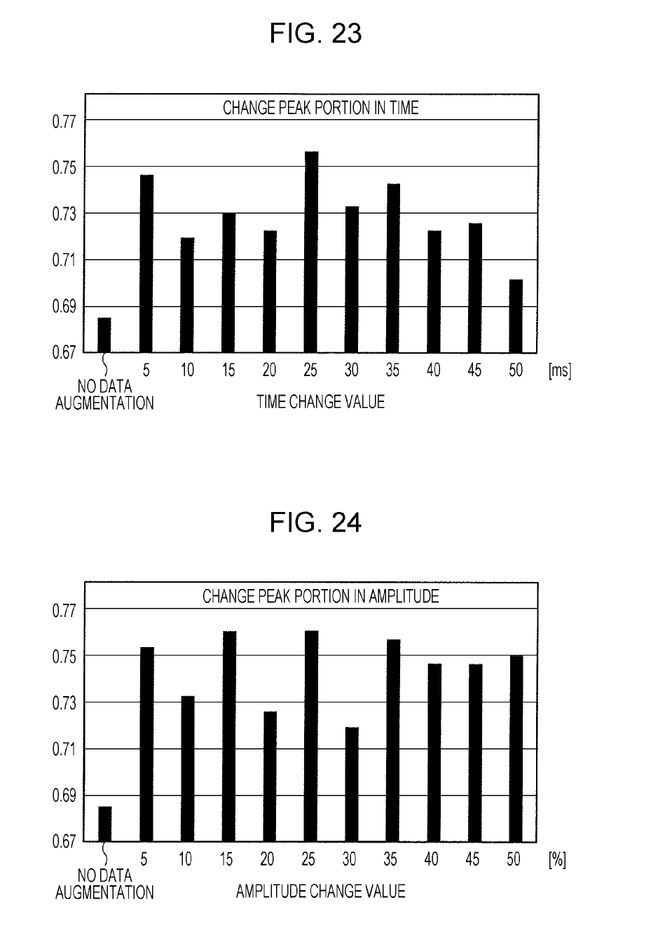

FIG. 23 illustrates the case where data augmentation is performed for a peak portion of the waveform in terms of time, which corresponds to the case C. As in FIG. 21, FIG. 23 illustrates the classification rates of classifiers generated using data items obtained by data augmentation performed using the time change value of “±n” ms. The classification rates of the classifiers in ten cases where “n” is equal to 5, 10, 15, 20, 25, 30, 35, 40, 45, and 50 are compared with the case where data augmentation is not performed on the brainwave data items of 20 trials. The numerals of the horizontal axis of FIG. 23 indicate the value of “n”.

FIG. 24 illustrates the case where data augmentation is performed for a peak portion of the waveform in terms of amplitude, which corresponds to the case D. FIG. 24 illustrates the classification rates of classifiers generated using data items obtained by data augmentation performed using the amplitude change values of “1+n/100” times and “1+2n/100” times for the data increasing ratio of ×3 in Table 3 above. The classification rates of the classifiers in ten cases where “n” is equal to 5, 10, 15, 20, 25, 30, 35, 40, 45, and 50 are compared with the case where data augmentation is not performed on the brainwave data items of 20 trials. The numerals of the horizontal axis of FIG. 24 indicate the value of “n”.

In all of FIGS. 21 to 24, the classification rates of the classifiers generated using the data items obtained by data augmentation exceed the classification rate of the classifier generated using the brainwave data items of 20 trials. In FIGS. 21 and 22, the classification rate tends to be low when the time change value and the amplitude change value are large; however this is not significant. In FIG. 23, the classification rate tends to be low when the time change value is large; however, this is not significant. In FIG. 24, the classification rate is not influenced by a change in the amplitude change value, and the classification rate is generally higher than those illustrated in FIGS. 21 to 23.

From the various experiments and analysis results described above, the classification accuracy is successfully increased in the case where a classifier is generated through machine learning using data items obtained by the data generation apparatus 1 through data augmentation, compared with a classifier obtained when data augmentation is not performed. In particular, the effect of improving the accuracy is large when the amount of data is small before data augmentation, and the data generation apparatus 1 is particularly effective in the case where it is difficult to obtain a large amount of data from a person.

In addition, in the case where the time change value is in a range from −250 ms to +250 ms in data augmentation, a classifier generated using data items whose amount is increased by data augmentation expectedly improves to have the same or higher performance than a classifier generated using original data before the amount is increased. In addition, in the case where the amplitude change value is in a range from -90% to +90% in data augmentation, that is, the multiplying factor is in a range from 0.1 times to 1.9 times, a classifier generated using data items whose amount is increased by data augmentation expectedly improves to have the same or higher performance than a classifier generated using original data before the amount is increased. In addition, the fact that the time change value within the range from −250 ms to +250 ms is effective on data augmentation and the fact that the amplitude change value within the range from 0.1 times to 1.9 times is effective on data augmentation can be qualitatively derived from results of hitherto conducted studies regarding a range of individual differences of brainwaves.

### Others

Although the data generation apparatus and the like according to one or more aspects have been described in accordance with the embodiments above, the present disclosure is not limited to these embodiments. Embodiments obtained by making various modifications conceivable by a person skilled in the art in the embodiments and embodiments obtained by combining elements of different embodiments together may also be within the scope of the one or more aspects as long as such embodiments do not depart from the essence of the present disclosure.

Although the effectiveness of the technique according to the present disclosure, such as the data generation apparatus and the classification system, has been described using brainwave data herein, the technique according to the present disclosure is effective in the case where event-related biological data other than brainwave data is measured. For example, in electrooculogram data that represents a bioelectric potential related to an eye movement, an event interval can be set using the start timing of the eye movement as the start point and data augmentation based on latency and amplitude can also be performed. In addition, in electromyogram data that represents a bioelectric potential related to a movement of a muscle, an event interval can be set using the timing at which the muscle starts moving or a stimulus is presented as the start point. Data augmentation can be performed using a period from the start point to a time point at which the largest potential difference is observed as the latency.

As described above, the technique according to the present disclosure may be implemented as a system, an apparatus, a method, an integrated circuit, a computer program, a computer-readable recording medium such as a recording disc, or any selective combination thereof. Examples of the computer-readable recording medium include a non-volatile recording medium, for example, a CD-ROM.

For example, individual processing units included in the above embodiments are typically implemented using large scale integration (LSI) which is an integrated circuit. These processing units may be formed as separate chips, or some or all of the processing units may be included in a chip.

Also, the circuit integration is not limited to LSI, and may be implemented using a dedicated circuit or general-purpose processor. A field programmable gate array (FPGA) that is programmable after manufacturing of the LSI or a reconfigurable processor in which connections and settings of circuit cells within the LSI are reconfigurable may be used.

In each of the embodiments described above, individual components may be implemented with dedicated hardware or by executing a software program suitable for the components. The individual components may be implemented as a result of a program execution unit such as a CPU or processor loading and executing a software program recorded on a recording medium, such as a hard disk or a semiconductor memory.

In addition, some or all of the components described above may be constituted by a removable integrated circuit (IC) card or a discrete module. The IC card or the module is a computer system including a microprocessor, a ROM, and a RAM. The IC card or the module may include an LSI or a system LSI mentioned above. The microprocessor operates in accordance with a computer program, whereby the IC card or the module achieves the function thereof. These IC card and module may be tamper-resistant.

The method according to the present disclosure may be implemented by a micro processing unit (MPU), a CPU, a processor, a circuit such as an LSI, an IC card, a discrete module, or the like.

Further, the technique according to the present disclosure may be implemented by a software program, a digital signal containing the software program, or a non-transitory computer-readable recording medium storing the software program. Needless to say, the computer program can be distributed via a communication medium, such as the Internet.

All the numerals representing the ordinal number, the quantity, etc. used above are merely examples used to describe the technique according to the present disclosure specifically, and the present disclosure is not limited to the numerals used as examples. In addition, connections between the components are merely examples used to describe the technique according to the present disclosure specifically, and connections that implement the functions of the present disclosure are not limited to these connections.

Moreover, division of functional blocks in each block diagram is merely an example. Thus, multiple functional blocks may be implemented as one functional block, one functional block may be divided into multiple functional blocks, or part of the function may be transferred to another functional block. In addition, functions of multiple functional blocks having similar functions may be processed by a single piece of hardware or software in parallel or in a time-division manner.

The technique according to the present disclosure is widely applicable to technologies that require a large amount of biological data items, such as biosignals.

What is claimed is:

1. A data generation apparatus comprising:
a memory that stores a generation rule including one or more time change values and/or one or more amplitude change values; and
a processor that
(a1) obtains a first biological data item including an event-related potential of a user and obtains a first label associated with the first biological data item,
(a2) refers to the generation rule and generates one or more second biological data items by changing the first biological data item using the one or more time change values and/or the one or more amplitude change values, and
(a3) outputs the one or more second biological data items associated with the first label, as training data related to the user.

2. The data generation apparatus according to claim 1, wherein the processor
(a4), before the (a2), obtains information on an event interval corresponding to the event-related potential and extracts a first time interval included in the first biological data item with reference to the information on the event interval, and
in the (a2), generates the one or more second biological data items by changing the first biological data item in the first time interval using the one or more time change values and/or the one or more amplitude change values.

3. The data generation apparatus according to claim 1, wherein
the first biological data item includes an event-related potential of a brainwave of the user,
the one or more time change values are values for changing a measurement time of the first biological data item in a range from −250 ms to +250 ms, and
the one or more amplitude change values are values for changing an amplitude of the first biological data item in a range from 0.1 times to 1.9 times.

4. A biological data measurement system comprising:
the data a generation apparatus according to claim 1; and
a biological data measurer that is attached to the user and that measures the first biological data item from the user.

5. A classifier generation apparatus comprising:
a memory that stores a generation rule including one or more time change values and/or one or more amplitude change values; and
a processor that
(b1) obtains a first biological data item including an event-related potential of a user, a third biological data item including an event-related potential of the user, a first label associated with the first biological data item, and a second label associated with the third biological data item,
(b2) refers to the generation rule and generates one or more second biological data items and one or more fourth biological data items by respectively changing the first biological data item and the third biological data item using the one or more time change values and/or the one or more amplitude change values, and
(b3) generates a classifier using training data that includes the first biological data item, the one or more second biological data items, the third biological data item, and the one or more fourth biological data items.

6. The classifier generation apparatus according to claim 5, wherein
the processor
(b4), before the (b2), obtains information on event intervals corresponding to the respective event-related potentials and extracts a first time interval and a third time interval included respectively in the first biological data item and the third biological data item with reference to the information on the event intervals, and
in the (b2), generates the one or more second biological data items and the one or more fourth biological data items by respectively changing the first biological data item in the first time interval and the third biological data item in the third time interval using the one or more time change values and/or the one or more amplitude change values.

7. The classifier generation apparatus according to claim 5, wherein
each of the first biological data item and the third biological data item includes an event-related potential of a brainwave of the user,
the one or more time change values are values for changing a measurement time of each of the first biological data item and the third biological data item in a range from −250 ms to +250 ms, and
the one or more amplitude change values are values for changing an amplitude of each of the first biological data item and the third biological data item in a range from 0.1 times to 1.9 times.

8. A data generation method comprising:
(a1) obtaining a first biological data item including an event-related potential of a user and obtaining a first label associated with the first biological data item;
(a2) referring to a generation rule that is stored in a memory and that includes one or more time change values and/or one or more amplitude change values and generating one or more second biological data items by changing the first biological data item using the one or more time change values and/or the one or more amplitude change values; and
(a3) outputting the one or more second biological data items associated with the first label, as training data related to the user,
at least one of the (a1), the (a2), and the (a3) being performed by a processor.

9. The data generation method according to claim 8, further comprising:
(a4), before the (a2), obtaining information on an event interval corresponding to the event-related potential and extracting a first time interval included in the first biological data item with reference to the information on the event interval, wherein
in the (a2), the one or more second biological data items are generated by changing the first biological data item in the first time interval using the one or more time change values and/or the one or more amplitude change values.

10. The data generation method according to claim 8, wherein
the first biological data item includes an event-related potential of a brainwave of the user,
the one or more time change values are values for changing a measurement time of the first biological data item in a range from −250 ms to +250 ms, and
the one or more amplitude change values are values for changing an amplitude of the first biological data item in a range from 0.1 times to 1.9 times.

11. A classifier generation method comprising:
(b1) obtaining a first biological data item including an event-related potential of a user, a third biological data item including an event-related potential of the user, a first label associated with the first biological data item, and a second label associated with the third biological data item;
(b2) referring to a generation rule that is stored in a memory and that includes one or more time change values and/or one or more amplitude change values and generating one or more second biological data items and one or more fourth biological data items by respectively changing the first biological data item and the third biological data item using the one or more time change values and/or the one or more amplitude change values; and
(b3) generating a classifier using training data that includes the first biological data item, the one or more second biological data items, the third biological data item, and the one or more fourth biological data items,
at least one of the (b1), the (b2), and the (b3) being performed by a processor.

12. The classifier generation method according to claim 11, further comprising:
(b4), before the (b2), obtaining information on event intervals corresponding to the respective event-related potentials and extracting a first time interval and a third time interval respectively included in the first biological data item and the third biological data item with reference to the information on the event intervals, wherein
in the (b2), the one or more second biological data items and the one or more fourth biological data items are generated by respectively changing the first biological data item in the first time interval and the third biological data item in the third time interval using the one or more time change values and/or the one or more amplitude change values.

13. The classifier generation method according to claim 11, wherein
each of the first biological data item and the third biological data item includes an event-related potential of a brainwave of the user,
the one or more time change values are values for changing a measurement time of each of the first biological data item and the third biological data item in a range from −250 ms to +250 ms, and
the one or more amplitude change values are values for changing an amplitude of each of the first biological data item and the third biological data item in a range from 0.1 times to 1.9 times.

14. A recording medium storing a control program causing a device including a processor to perform a process, the recording medium being non-volatile and computer-readable, the process comprising:
(a1) obtaining a first biological data item including an event-related potential of a user and obtaining a first label associated with the first biological data item;
(a2) referring to a generation rule that is stored in a memory and that includes one or more time change values and/or one or more amplitude change values and generating one or more second biological data items by changing the first biological data item using the one or more time change values and/or the one or more amplitude change values; and
(a3) outputting the one or more second biological data items associated with the first label, as training data related to the user.

15. The recording medium according to claim 14, the process further comprising:
(a4), before the (a2), obtaining information on an event interval corresponding to the event-related potential and extracting a first time interval included in the first biological data item with reference to the information on the event interval, wherein
in the (a2), the one or more second biological data items are generated by changing the first biological data in the first time interval using the one or more time change values and/or the one or more amplitude change values.

16. The recording medium according to claim 14, wherein
the first biological data item includes an event-related potential of a brainwave of the user,
the one or more time change values are values for changing a measurement time of the first biological data item in a range from −250 ms to +250 ms, and
the one or more amplitude change values are values for changing an amplitude of the first biological data item in a range from 0.1 times to 1.9 times.

17. A recording medium storing a control program causing a device including a processor to perform a process, the recording medium being non-volatile and computer-readable, the process comprising:
(b1) obtaining a first biological data item including an event-related potential of a user, a third biological data item including an event-related potential of the user, a first label associated with the first biological data item, and a second label associated with the third biological data item;
(b2) referring to a generation rule that is stored in a memory and that includes one or more time change values and/or one or more amplitude change values and generating one or more second biological data items and one or more fourth biological data items by respectively changing the first biological data item and the third biological data item using the one or more time change values and/or the one or more amplitude change values; and
(b3) generating a classifier using training data that includes the first biological data item, the one or more second biological data items, the third biological data item, and the one or more fourth biological data items.

18. The recording medium according to claim 17, the process further comprising:
(b4), before the (b2), obtaining information on event intervals corresponding to the respective event-related potentials and extracting a first time interval and a third time interval respectively included in the first biological data item and the third biological data item with reference to the information on the event intervals, wherein
in the (b2), the one or more second biological data items and the one or more fourth biological data items are generated by respectively changing the first biological data item in the first time interval and the third biological data item in the third time interval using the one or more time change values and/or the one or more amplitude change values.

19. The recording medium according to claim 17, wherein
each of the first biological data item and the third biological data item includes an event-related potential of a brainwave of the user,
the one or more time change values are values for changing a measurement time of each of the first biological data item and the third biological data item in a range from −250 ms to +250 ms, and
the one or more amplitude change values are values for changing an amplitude of each of the first biological data item and the third biological data item in a range from 0.1 times to 1.9 times.
